(19) United States
(12) Patent Application Publication
(10) Pub. No.: US 2017/0113993 A1
(43) Pub. Date: Apr. 27, 2017

(54) ORGANIC ACIDS FROM HOMOCITRATE AND HOMOCITRATE DERIVATIVES

(71) Applicant: BIOAMBER INC., St. Paul, MN (US)

(72) Inventors: Ralph Thomas BAKER, Ottawa (CA); James F. WHITE, Richland, WA (US); Leo E. MANZER, Wilmington, DE (US); Spyridon NTAIS, Ottawa (CA); Olena BARANOVA, Ottawa (CA); Indira THAPA, Saint Louis Park, MN (US); Man Kit LAU, Minneapolis, MN (US); Cathy Staloch HASS, Saint Louis Park, MN (US)

(21) Appl. No.: 15/317,267

(22) PCT Filed: Jun. 10, 2015
(86) PCT No.: PCT/US15/35178

§ 371 Date: Dec. 8, 2016

Related U.S. Application Data
(60) Provisional application No. 62/010,371, filed on Jun. 10, 2014.

(51) Int. Cl. C07C 51/38; C12R 1/645; B01J 27/053; B01J 23/44; B01J 27/232; C07C 51/31; B01J 21/18

(52) U.S. Cl. CPC C07C 51/38; C07C 51/31; C12R 1/645; B01J 21/18; B01J 23/44; B01J 27/232; B01J 27/053

(57) ABSTRACT

This disclosure relates to methods for converting homocitric acid to adipic acid, and more particularly to methods of using metal catalysts to catalyze the conversion of homocitric acid to adipic acid.

### CROSS-REFERENCE TO RELATED APPLICATIONS

This application claims priority to U.S. Provisional Application Ser. No. 62/010,371, filed on Jun. 10, 2014, the contents of which are hereby incorporated by reference in their entirety.

### TECHNICAL FIELD

This disclosure relates to methods for converting homocitric acid or derivatives of homocitrate to organic acids, including to adipic acid.

### BACKGROUND

Currently, many carbon containing chemicals are derived from petroleum based sources. Reliance on petroleum-derived feedstocks contributes to depletion of petroleum reserves and the harmful environmental impact associated with oil drilling.

Certain carbonaceous products of sugar fermentation are seen as replacements for petroleum-derived materials that are used for the manufacture of carbon-containing chemicals, such as polymers. Such products include, for example, diacids and triacids that are used to make polymers. A particular example of a useful diacid is adipic acid. Adipic acid represents a large market for which all commercial production today is petroleum-derived.

### SUMMARY

Provided herein are compositions comprising diacids and triacids that can be made using the disclosed methods. The methods described allow, inter alia, for the creation of compositions containing the compounds shown in Formulas I, IV, V and VI, below. In some instances the compositions containing one or more of the compounds shown in Formulas I, IV, V and VI can be subjected to a separation step so that the composition contains greater than 80%, 90%, 95%, 96%, 97%, 98%, 99%, or 99.5% of one of the compounds in Formulas I, IV, V and VI. One of ordinary skill in the art will appreciate that such separation can be accomplished using extraction, distillation and/or crystallization.

Provided herein is a method for making adipic acid, or a salt or ester thereof, the method comprising contacting homocitric acid, or a salt, ester, or lactone thereof, or homoaconitic acid, or a salt or ester, thereof, with a metal catalyst.

A method for making a compound of Formula I:

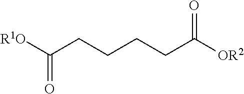

or a salt thereof,
wherein:
each R¹ and R² is individually selected from H and a protecting group is also provided. The method comprising contacting a metal catalyst with a composition comprising a compound of Formula II:

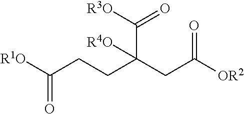

or a salt thereof,
wherein:
each R¹, R², R³, and R⁴ is individually selected from H and a protecting group. Also provided herein is a method for making a compound of Formula I, or a salt thereof, that includes contacting a metal catalyst with composition comprising a compound of Formula III:

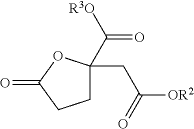

or a salt thereof,
wherein:
each R² and R³ is individually selected from H and a protecting group.

In some embodiments, a compound of Formula I, or a salt thereof, can be prepared by a) hydrogenolysis of a compound of Formula II, or a salt thereof, to prepare a compound of Formula IV:

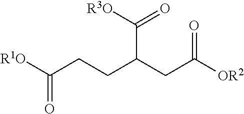

or a salt thereof,
wherein:
each R¹, R², R³, and R⁴ is individually selected from H and a protecting group; and b) selective decarboxylation of the compound of Formula IV to make a compound of Formula I, or a salt thereof.

In some embodiments, a compound of Formula I, or a salt thereof, can be prepared by a) hydrogenolysis of a compound of Formula III, or a salt thereof, to prepare a compound of Formula IV, or a salt thereof; and b) selective decarboxylation of the compound of Formula IV to make a compound of Formula I, or a salt thereof.

In some embodiments, a method for making adipic acid, or a salt or ester thereof, can include contacting homocitric acid lactone with a Pd(S)/C catalyst. For example, a compound of Formula I, or a salt thereof, can be prepared using a method comprising contacting a Pd(S)/C catalyst with composition comprising a compound of Formula III, or a salt thereof.

Also provided herein is a method for making 2-ethylsuccinic acid, or a salt or ester thereof, the method comprising contacting homocitric acid, or a salt, ester, or lactone thereof, with a metal catalyst.

A method for making a compound of Formula V:

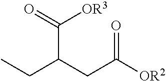

or a salt thereof,
wherein:
each R² and R³ is individually selected from H and a protecting group is also provided. The method can include contacting a metal catalyst with a composition comprising a compound of Formula II, or a salt thereof, and/or a compound of Formula III, or a salt thereof

In some embodiments, a compound of Formula V, or a salt thereof, can be prepared by a method comprising hydrogenolysis of a compound of Formula II, or a salt thereof, and/or a compound of Formula III, or a salt thereof, to prepare a compound of Formula IV, or a salt thereof; and b) selective decarboxylation of the compound of Formula IV to make a compound of Formula V, or a salt thereof.

Further provided herein is a method for making 2-methylpentanedioic acid, or a salt or ester thereof, the method comprising contacting homocitric acid, or a salt, ester, or lactone thereof, with a metal catalyst.

A method for making a compound of Formula VI:

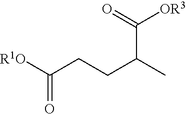

or a salt thereof,
wherein:
each R¹ and R³ is individually selected from H and a protecting group is also provided. The method can include contacting a metal catalyst with a composition comprising a compound of Formula II, or a salt thereof, and/or a compound of Formula III, or a salt thereof.

In some embodiments, a compound of Formula V, or a salt thereof, can be prepared by a method comprising hydrogenolysis of a compound of Formula II, or a salt thereof, and/or a compound of Formula III, or a salt thereof, to prepare a compound of Formula IV, or a salt thereof; and b) selective decarboxylation of the compound of Formula IV to make a compound of Formula V, or a salt thereof.

This disclosure provides a method for making a composition comprising two or more compounds selected from the group consisting of: adipic acid, 1,2,4-butanetricarboxylic acid, 2-ethylsuccinic acid, and 2-methylpentanedioic acid, or a salt or ester thereof, the method comprising contacting homocitric acid, or a salt, ester, or lactone thereof, with a metal catalyst.

In some embodiments, a method for making a composition comprising two or more compounds selected from the group consisting of:

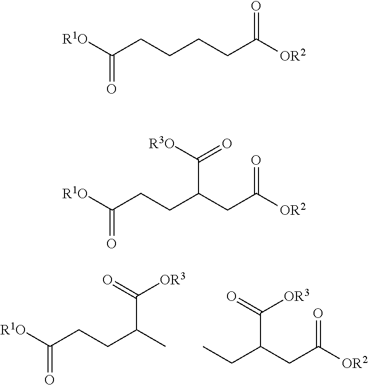

or a salt thereof,
wherein:
each R¹, R², and R³ is individually selected from H and a protecting group; comprises contacting a metal catalyst with a composition comprising a compound of Formula II, or a salt thereof, and/or a compound of Formula III, or a salt thereof.

In some embodiments, a composition comprising two or more compounds selected from Formula I, IV, V, and VI, or a salt thereof, can be prepared by a method comprising hydrogenolysis of a compound of Formula II, or a salt thereof, and/or a compound of Formula III, or a salt thereof, to prepare a compound of Formula IV, or a salt thereof; and b) selective decarboxylation of the compound of Formula IV to the composition.

In some of the methods described herein, the metal catalyst is a heterogeneous catalyst. In some embodiments, the metal catalyst comprises a metal selected from the group consisting of Ni, Pd, Pt, Re, Au, Ag, Cu, Zn, Rh, Ru, Bi, Fe, Co, Os, Ir, V, and mixtures of two or more thereof. For example, the metal catalyst comprises a metal selected from the group consisting of Pd and Pt. In some embodiments, the metal catalyst comprises Pd. In some embodiments, the metal catalyst is a supported catalyst. In some embodiments, the metal catalyst comprises a promoter. For example, the promoter comprises sulfur.

In some embodiments, the method is performed at a temperature of at least about 100° C. For example, the method is performed at a temperature of about 100° C. to about 200° C. For example, the method is performed at a temperature of about 150° C. to about 300° C. In some embodiments, the method is performed at a temperature of about 150° C. to about 180° C.

In some embodiments, the metal catalyst is activated prior to the contacting. For example, the metal catalyst is activated under hydrogen gas, inert gas or a combination of inert gas and hydrogen. In some embodiments, the metal catalyst is activated at a temperature of about 100° C. to about 200° C., 200 to about 300° C., or 300° C. to about 400° C.

Also provided herein is a composition comprising two or more compounds selected from the group consisting of: adipic acid, 1,2,4-butanetricarboxylic acid, 2-ethylsuccinic acid, and 2-methylpentanedioic acid, or a salt or ester thereof. In some embodiments, a composition can comprise two or more compounds selected from the group consisting of:

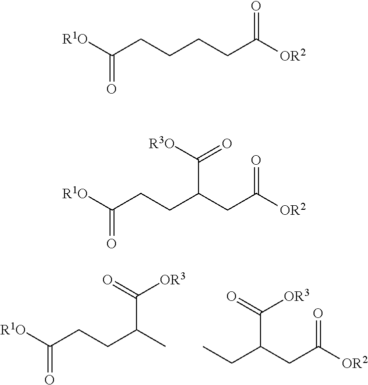

or a salt thereof,
wherein:
each R¹, R², and R³ is individually selected from H and a protecting group.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF THE DRAWINGS

FIG. 1 is a GC/MS chromatogram of pure lactone (no catalyst) before (black line) and after hydrogenolysis reaction (blue line).

FIG. 2 shows GC/MS chromatograms of the blank sample (lactone after hydrogenolysis without catalyst) and of the samples using catalysts No 7, 9, 13, 51, 53, 54.

FIG. 3 shows GC/MS chromatograms of the control sample (lactone after hydrogenolysis without catalyst) and of the homocitric acid lactone samples using catalysts No. 6 and 59.

FIG. 4 shows GC/MS chromatograms of the homocitric acid lactone samples using catalyst No. 51 activated by all three methods.

FIG. 5 shows GC/MS chromatograms of the control sample (without catalyst) and samples using catalyst No. 6 with and without addition of 1, 2 and 3 equivalents of NaOH.

FIG. 6 shows GC/MS chromatograms of the control sample (lactone without catalyst) and samples using catalyst No. 59 with and without addition of 1, 2 and 3 equivalents of NaOH.

FIG. 7 shows GC/MS chromatograms of the blank sample (lactone after hydrogenolysis without catalyst) and of the samples using catalysts Nos. 7, 9, 13, 51, 53, 54.

FIG. 8 shows GC/MS chromatograms of lactone after hydrogenolysis without catalyst and of the samples using catalysts Nos. 7 and 51 (Method C) and the commercial dry/reduced catalyst No. 59.

FIG. 9 illustrates quantitative conversion of homocitric acid lactone with catalyst No. 13.

FIG. 10 shows an exemplary chromatogram including decarboxylation products.

FIG. 11 illustrates conversion of 1,2,4-butantricarboxylic acid to adipic acid.

FIG. 12 illustrates the reaction products with Pt/C and Pt(S)/C catalysts.

FIG. 13 is a GCFID chromatogram (after methyl ester derivatization) for conversion of homocitric acid lactone to adipic acid.

FIG. 14 is a GCFID chromatogram (after methyl ester derivatization) for conversion of homocitric acid lactone to adipic acid.

FIG. 15 shows conversion of homocitric acid lactone to adipic acid under N₂. Where 2ES=2-ethylsuccinate (blue bar, first from the left), 2MG=2-methylglutarate (red bar, second from the left), AA=Adipate (green bar, third from the left), TA=1,2,4-butanetricarboxylate (purple bar, fourth from the left).

FIG. 16 shows conversion of homocitric acid lactone to adipic acid under mixed N₂/H₂, H₂ and N₂ pressure. Where 2ES=2-ethylsuccinate (blue bar, bottom), 2MG=2-methylglutarate (red bar, second from the bottom), AA=Adipate (green bar, third from the bottom), TA=1,2,4-butanetricarboxylate (purple bar, fourth from the bottom).

FIG. 17 shows conversion of homocitric acid lactone to adipic acid in water/DMSO (50:50) solvent. Where 2ES=2-ethylsuccinate (blue bar, first from the left), AA=Adipate (red bar, second from the left), TA=1,2,4-butanetricarboxylate (green bar, third from the left).

FIG. 18 is a GCFID chromatogram for conversion of homocitric acid lactone to adipic acid in an autoclave condition.

FIG. 19 shows the mol % concentration of the 4 main products: 2ES=2-ethylsuccinate (blue bar, bottom), 2MG=2-methylglutarate (red bar, second from the bottom), AA=Adipate (green bar, third from the bottom), TA=1,2,4-butanetricarboxylate (purple bar, fourth from the bottom).

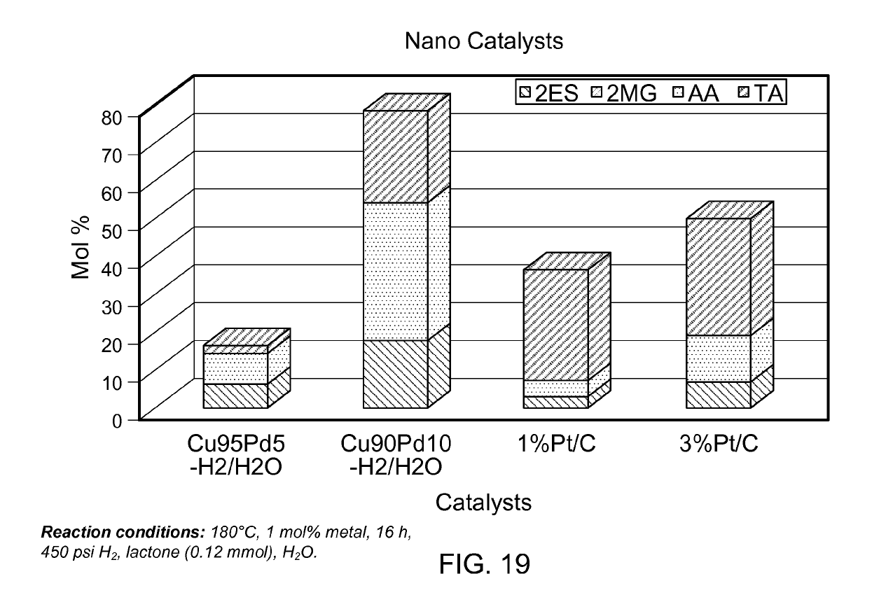

FIG. 20 illustrates conversion of homocitric acid lactone, homocitric acid, and homoaconitic acid to adipic acid under N₂. Where 2ES=2-ethylsuccinate (blue bar, bottom), 2MG=2-methylglutarate (red bar, second from the bottom), AA=Adipate (green bar, third from the bottom), TA=1,2,4-butanetricarboxylate (purple bar, fourth from the bottom).

### DETAILED DESCRIPTION

Provided herein are methods for making adipic acid (CH₂)₄(COOH)₂. Approximately 2.5 billion kilograms of this white crystalline powder are produced annually. Adipic acid is primarily used as a monomer for the production of nylon, but it is also involved in the production of polyurethane and its esters (adipates) are plasticizers used in the production of PVC. Accordingly, from an industrial perspective, it is considered to be one of the most important dicarboxylic acids.

The methods provided herein relate to the conversion of homocitric acid to adipic acid and related compounds 2-ethylsuccinic acid and 2-methylpentanedioic acid. For example, the preparation of adipic acid can be as shown in Scheme 1.

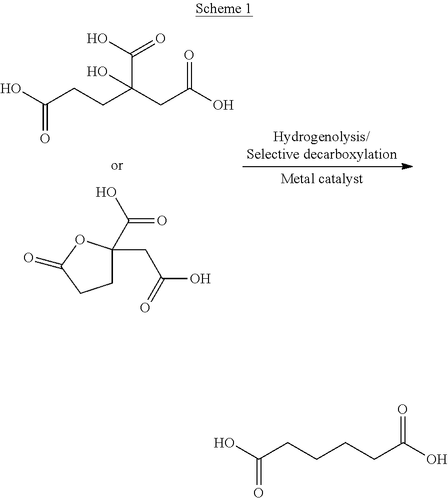 [SMILES: O=C(O)CCC(O)(CC(=O)O)C(=O)O ; O=C(O)CC1(C(=O)O)CCC(=O)O1 ; O=C(O)CCCCC(=O)O]

wherein each of the compounds may be present as a salt or ester thereof

Without being bound by theory, it is believed that the reaction proceeds as shown in Scheme 2.

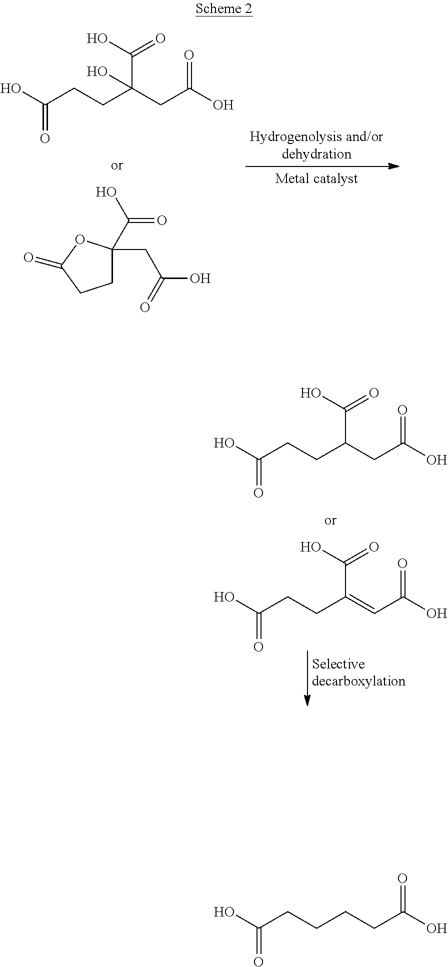 [SMILES: O=C(O)CCC(O)(CC(=O)O)C(=O)O ; O=C(O)CC1(C(=O)O)CCC(=O)O1 ; O=C(O)CCC(CC(=O)O)C(=O)O ; O=C(O)/C=C(/CCC(=O)O)C(=O)O ; O=C(O)CCCCC(=O)O]

wherein each of the compounds may be present as a salt or ester thereof.

Accordingly, provided herein are methods for making adipic acid, or a salt or ester thereof, the method comprising contacting homocitric acid, or a salt, ester, or lactone thereof, with a metal catalyst.

In some embodiments, a method for making a compound of Formula I:

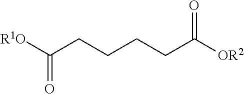

or a salt thereof,
wherein:
each R¹ and R² is individually selected from H and a protecting group is provided. The method comprising contacting a metal catalyst with a composition comprising a compound of Formula II:

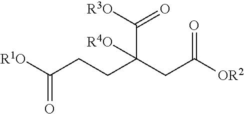

or a salt thereof,
wherein:
each R¹, R², R³, and R⁴ is individually selected from H and a protecting group. In some embodiments, a compound of Formula I, or a salt thereof, can be prepared by contacting a metal catalyst with composition comprising a compound of Formula III:

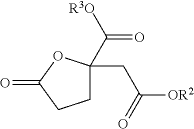

or a salt thereof,
wherein:
each R² and R³ is individually selected from H and a protecting group.

As shown in Scheme 2, it is thought that a compound of Formula I, or a salt thereof, can be prepared in some embodiments by a method comprising a) hydrogenolysis of a compound of Formula II, or a salt thereof, to prepare a compound of Formula IV:

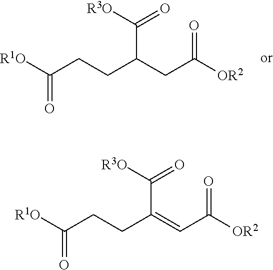

or a salt thereof,
wherein:
each R¹, R², R³, and R⁴ is individually selected from H and a protecting group; and b) selective decarboxylation of the compound of Formula IV to prepare a compound of Formula I, or a salt thereof. In some embodiments, a compound of Formula I, or a salt thereof, can be prepared by a method comprising dehydration and/or hydrogenolysis of a compound of Formula III, or a salt thereof, to prepare a compound of Formula IV, or a salt thereof, followed by selective decarboxylation of the compound of Formula IV to prepare a compound of Formula I, or a salt thereof.

This disclosure further provides a method for making adipic acid, or a salt or ester thereof, the method comprising contacting homocitric acid lactone with a Pd(S)/C catalyst. In some embodiments, a method for making a compound of Formula I, or a salt thereof, includes contacting a Pd(S)/C catalyst with composition comprising a compound of Formula III, or a salt thereof. For example, a method for making a compound of Formula I, or a salt thereof, can include hydrogenolysis of a compound of Formula III, or a salt thereof, to prepare a compound of Formula IV, followed by selective decarboxylation of the compound of Formula IV to make a compound of Formula I, or a salt thereof. In some embodiments, such a method is performed in a single reaction pot in the presence of a Pd(S)/C catalyst.

Also provided herein are methods for making 2-ethylsuccinic acid, or a salt or ester thereof. The methods can include contacting homocitric acid, or a salt, ester, or lactone thereof, with a metal catalyst. In some embodiments, a method for making a compound of Formula V:

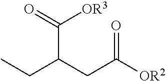

or a salt thereof,
wherein:
each R² and R³ is individually selected from H and a protecting group is provided. The method comprising contacting a metal catalyst with a composition comprising a compound of Formula II, or a salt thereof, and/or a compound of Formula III, or a salt thereof.

In some embodiments, a method for making a compound of Formula V, or a salt thereof, can include hydrogenolysis of a compound of Formula II, or a salt thereof, and/or a compound of Formula III, or a salt thereof, to prepare a compound of Formula IV, or a salt thereof, followed by selective decarboxylation of the compound of Formula IV to make a compound of Formula V, or a salt thereof.

Further provided herein is a method for making 2-methylpentanedioic acid, or a salt or ester thereof, the method comprising contacting homocitric acid, or a salt, ester, or lactone thereof, with a metal catalyst. In some embodiments, a method for making a compound of Formula VI:

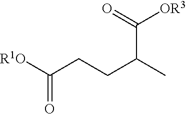

or a salt thereof,
wherein:
each R¹ and R³ is individually selected from H and a protecting group is provided. The method comprising contacting a metal catalyst with a composition comprising a compound of Formula II, or a salt thereof, and/or a compound of Formula III, or a salt thereof.

In some embodiments, a method for making a compound of Formula VI, or a salt thereof, can include hydrogenolysis of a compound of Formula II, or a salt thereof, and/or a compound of Formula III, or a salt thereof, to prepare a compound of Formula IV, or a salt thereof, followed by selective decarboxylation of the compound of Formula IV to make a compound of Formula VI, or a salt thereof.

The methods provided herein can be used to prepare one or more of the compounds described herein. For example, the methods described herein can be used to prepare a composition comprising two or more compounds selected from the group consisting of: adipic acid, 1,2,4-butanetricarboxylic acid, 2-ethylsuccinic acid, and 2-methylpentanedioic acid, or a salt or ester thereof. In some embodiments, the method comprises contacting homocitric acid, or a salt, ester, or lactone thereof, with a metal catalyst. In some embodiments, a method is provided for making a composition comprising two or more compounds selected from the group consisting of:

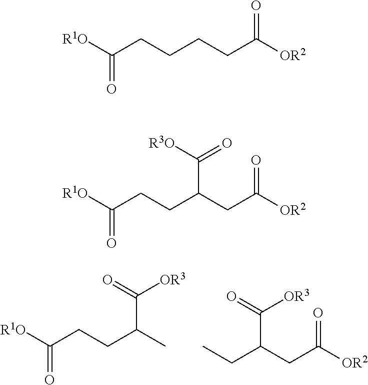

or a salt thereof,
wherein:
each R¹, R², and R³ is individually selected from H and a protecting group; the method comprising contacting a metal catalyst with a composition comprising a compound of Formula II, or a salt thereof, and/or a compound of Formula III, or a salt thereof.

In some embodiments, a method for making a composition comprising two or more compounds of Formula I, IV, V, and VI, or a salt thereof, can include hydrogenolysis of a compound of Formula II, or a salt thereof, and/or a compound of Formula III, or a salt thereof, to prepare a compound of Formula IV, or a salt thereof, followed by selective decarboxylation of the compound of Formula IV to the composition.

In the compounds described above (i.e., compounds of Formula I, II, III, IV, V, and/or IV), reference is made to a protecting group. In some embodiments, a carboxyl group may be protected (e.g., in the case of R¹, R², and R³). For this purpose, R¹, R², and R³ may include any suitable carboxyl protecting group including, but not limited to, esters, amides, or hydrazine protecting groups. Each occurrence of the protecting group may be the same or different.

In particular, the ester protecting group may include methyl, ethyl, methoxy methyl (MOM), benzyloxymethyl (BOM), methoxyethoxymethyl (MEM), 2-(trimethylsilyl)ethoxymethyl (SEM), methylthiomethyl (MTM), phenylthiomethyl (PTM), azidomethyl, cyanomethyl, 2,2-dichloro-1,1-difluoroethyl, 2-chloroethyl, 2-bromoethyl, tetrahydropyranyl (THP), 1-ethoxyethyl (EE), phenacyl, 4-bromophenacyl, cyclopropylmethyl, allyl, propargyl, isopropyl, cyclohexyl, t-butyl, benzyl, 2,6-dimethylbenzyl, 4-methoxybenzyl (MPM-OAr), o-nitrobenzyl, 2,6-dichlorobenzyl, 3,4-dichlorobenzyl, 4-(dimethylamino)carbonylbenzyl, 4-methylsulfinylbenzyl (Msib), 9-anthrylmethyl, 4-picolyl, heptafluoro-p-tolyl, tetrafluoro-4-pyridyl, trimethylsilyl (TMS), t-butyldimethylsilyl (TBDMS), t-butyldiphenylsilyl (TBDPS), and triisopropylsilyl (TIPS) protecting groups.

The amide and hydrazine protecting groups may include N,N-dimethylamide, N-7-nitroindoylamide, hydrazide, N-phenylhydrazide, and N,N′-diisopropylhydrazide.

In some embodiments, a hydroxyl group may be protected (e.g., in the case of R⁴). For this purpose, R⁴ may include any suitable hydroxyl protecting group including, but not limited to, ether, ester, carbonate, or sulfonate protecting groups. Each occurrence of the protecting group may be the same or different.

In particular, the ether protecting group may include methyl, methoxy methyl (MOM), benzyloxymethyl (BOM), methoxyethoxymethyl (MEM), 2-(trimethylsilyl)ethoxymethyl (SEM), methylthiomethyl (MTM), phenylthiomethyl (PTM), azidomethyl, cyanomethyl, 2,2-dichloro-1,1-difluoroethyl, 2-chloroethyl, 2-bromoethyl, tetrahydropyranyl (THP), 1-ethoxyethyl (EE), phenacyl, 4-bromophenacyl, cyclopropylmethyl, allyl, propargyl, isopropyl, cyclohexyl, t-butyl, benzyl, 2,6-dimethylbenzyl, 4-methoxybenzyl (MPM-OAr), o-nitrobenzyl, 2,6-dichlorobenzyl, 3,4-dichlorobenzyl, 4-(dimethylamino)carbonylbenzyl, 4-methylsulfinylbenzyl (Msib), 9-anthrylemethyl, 4-picolyl, heptafluoro-p-tolyl, tetrafluoro-4-pyridyl, trimethylsilyl (TMS), t-butyldimethylsilyl (TBDMS), t-butyldiphenylsilyl (TBDPS), and triisopropylsilyl (TIPS) protecting groups.

The ester protecting group may include acetoxy (OAc), aryl formate, aryl acetate, aryl levulinate, aryl pivaloate, aryl benzoate, and aryl 9-fluoroenecarboxylate. In one embodiment, the ester protecting group is an acetoxy group.

The carbonate protecting group may include aryl methyl carbonate, 1-adamantyl carbonate (Adoc-OAr), t-butyl carbonate (BOC-OAr), 4-methylsulfinylbenzyl carbonate (Msz-OAr), 2,4-dimethylpent-3-yl carbonate (Doc-OAr), aryl 2,2,2-trichloroethyl carbonate, aryl vinyl carbonate, aryl benzyl carbonate, and aryl carbamate.

The sulfonate protecting groups may include aryl methanesulfonate, aryl toluenesulfonate, and aryl 2-formylbenzenesulfonate.

Preparation of compounds as described herein can involve the protection and deprotection of various chemical groups. The need for protection and deprotection, and the selection of appropriate protecting groups, can be readily determined by one skilled in the art. The chemistry of protecting groups can be found, for example, in Protecting Group Chemistry, 1^{st} Ed., Oxford University Press, 2000; March's Advanced Organic chemistry: Reactions, Mechanisms, and Structure, 5^{th} Ed., Wiley-Interscience Publication, 2001; and Peturssion, S. et al., “Protecting Groups in Carbohydrate Chemistry,” J. Chem. Educ., 74(11), 1297 (1997) (each of which is incorporated herein by reference in their entirety.

In the methods described above, homocitric acid, or a salt, ester, or lactone thereof, may be obtained by methods known by those of ordinary skill in the art. For example, the homocitric acid, or a salt, ester, or lactone thereof, may be obtained commercially or may be produced synthetically. In some embodiments, the homocitric acid, or a salt, ester, or lactone thereof, may be prepared using fermentation methods such as those described in WO 2014/043182, which is incorporated by reference in its entirety herein.

A metal catalyst as used herein can include any suitable metal catalyst. For example, a suitable metal catalyst would include on that could facilitate the conversion of homocitric acid, or a salt, ester, or lactone thereof, to one or more of adipic acid, 1,2,4-butanetricarboxylic acid, 2-ethylsuccinic acid, and 2-methylpentanedioic acid, or a salt or ester thereof.

In some embodiments, a suitable metal catalyst for the present methods is a heterogeneous (or solid) catalyst. The metal catalyst (e.g., a heterogeneous catalyst) can be supported on at least one catalyst support (referred to herein as “supported metal catalyst”). When used, the at least one support for a metal catalyst can be any solid substance that is inert under the reaction conditions including, but not limited to, oxides such as silica, alumina and titania, compounds thereof or combinations thereof; barium sulfate; zirconia; carbons (e.g., acid washed carbon); and combinations thereof. Acid washed carbon is a carbon that has been washed with an acid, such as nitric acid, sulfuric acid or acetic acid, to remove impurities. The support can be in the form of powders, granules, pellets, or the like. The supported metal catalyst can be prepared by depositing the metal catalyst on the support by any number of methods well known to those skilled in the art, such as spraying, soaking or physical mixing, followed by drying, calcination, and if necessary, activation through methods such as heating, reduction, and/or oxidation. In some embodiments, activation of the catalyst can be performed in the presence of hydrogen gas. For example, the activation can be performed under hydrogen flow or pressure (e.g., a hydrogen pressure of about 200 psi). In some embodiments, the metal catalyst is activated at a temperature of about 100° C. to about 500° C. (e.g., about 100° C. to about 500° C.).

In some embodiments, the loading of the at least one metal catalyst on the at least one support is from about 0.1 weight percent to about 20 weight percent based on the combined weights of the at least one acid catalyst plus the at least one support. For example, the loading of the at least one metal catalyst on the at least one support can be about 5% by weight. In some embodiments, the loading of the at least one metal catalyst on the at least one support can be about 1% to about 10% by weight (e.g., about 1%, about 3%, about 5%, or about 10%).

A metal catalyst can include a metal selected from nickel, palladium, platinum, copper, zinc, rhodium, ruthenium, bismuth, iron, cobalt, osmium, iridium, vanadium, and combinations of two or more thereof. In some embodiments, the metal catalyst comprises palladium or platinum. For example, the metal catalyst can comprise palladium. In some embodiments, the metal catalyst is a bimetallic catalyst. For example, the metal catalyst can include palladium and copper. The atomic ratio of the two metals can range from about 99:1 to about 80:20 (e.g., 95:5, 90:10, 85:15).

In some embodiments, the metal catalyst can be a nanocatalyst. For example, the metal catalyst can be prepared in the form of nanoparticles (see, for example, Example 7). In some embodiments, the nanocatalyst comprises palladium or platinum. For example, the nanocatalyst can comprise palladium. In some embodiments, the nanocatalyst is a bimetallic catalyst. For example, the nanocatalyst can include palladium and copper. The atomic ratio of the two metals can range from about 99:1 to about 80:20 (e.g., 95:5, 90:10, 85:15). Nanocatalysts can be used alone (unsupported) or as supported nanocatalysts. For example, the nanoparticles can be prepared as carbon supported nanocatalysts.

Unsupported catalyst can also be used. A catalyst that is not supported on a catalyst support material is an unsupported catalyst. An unsupported catalyst may be platinum black or a RANEY® (W.R. Grace & Co., Columbia, Md.) catalyst, for example (Ber. (1920) V53 pp 2306, JACS (1923) V45, 3029 and USA 2955133). RANEY® catalysts have a high surface area due to selectively leaching an alloy containing the active metal(s) and a leachable metal (usually aluminum). RANEY® catalysts have high activity due to the higher specific area and allow the use of lower temperatures in hydrogenation reactions. The active metals of RANEY® catalysts include nickel, copper, cobalt, iron, rhodium, ruthenium, rhenium, osmium, iridium, platinum, palladium, compounds thereof and combinations thereof.

Promoter metals may also be added to the base RANEY® metals to affect selectivity and/or activity of the RANEY® catalyst. Promoter metals for RANEY® catalysts may be selected from transition metals from Groups IIIA through VIIIA, IB and IIB of the Periodic Table of the Elements. Examples of promoter metals include chromium, cobalt, molybdenum, platinum, rhodium, ruthenium, osmium, and palladium, typically at about 2% by weight of the total RANEY metal. The method of using the catalyst to hydrogenate a feed can be performed by various modes of operation generally known in the art. Thus, the overall hydrogenation process can be performed with a fixed bed reactor, various types of agitated slurry reactors, either gas or mechanically agitated, or the like. The hydrogenation process can be operated in either a batch or continuous mode, wherein an aqueous liquid phase containing the precursor to hydrogenate is in contact with gaseous phase containing hydrogen at elevated pressure and the particulate solid catalyst.

A chemical promoter can be used to augment the activity of the catalyst. The promoter can be incorporated into the catalyst during any step in the chemical processing of the catalyst constituent. The chemical promoter generally enhances the physical or chemical function of the catalyst agent, but can also be added to retard undesirable side reactions. Suitable promoters include, for example, sulfur (e.g., sulfide) and phosphorous (e.g., phosphate). In some embodiments, the promoter comprises sulfur.

Non-limiting examples of suitable metal catalysts as described herein are provided in Table 1.

TABLE 1

| A/a | Product | Description | Company | Batch No |
|---|---|---|---|---|
| 1 | RANEY Ni 4.2 | Ni Catalyst | W.R. Grace | NA |
| 2 | Cu-0860 | Cu Catalyst | BASF | NA- |
| | E 1/16″ 3F | (unreduced as oxide) | | |
| 3 | Cu-0865 | Cu Catalyst | BASF | NA- |
| | T 3/16″ | (unreduced as oxide) | | |
| 4 | F51-8PPT | Cu/Zn/Al MeOH | Synetics | NA |
| | | (unreduced as oxides) | Johnson | |
| | | | Matthey | |
| | | | Catalysts | |
| 5 | 10% Pd/C | 10% Pd on Carbon | Johnson | |
| | A402028-10 | (51.47% H₂O) | Matthey | |
| | | | Catalysts | |
| 6 | 5% Pd/C | 5% Pd on Carbon | Johnson | |
| | A401102-5 | (56.34% H₂O) | Matthey | |
| | | | Catalysts | |
| 7 | 5% Pd/C | 5% Pd on Carbon | Johnson | |
| | A405028-5 | (47.22% H₂O) | Matthey | |
| | | | Catalysts | |
| 8 | 5% Pd/C | 5% Pd on Carbon | Johnson | |
| | A405032-5 | (67.86% H₂O) | Matthey | |
| | | | Catalysts | |
| 9 | 5% Pd/C | 5% Pd on Carbon | Johnson | |
| | A405038-5 | (64.81% H₂O) | Matthey | |
| | | | Catalysts | |
| 10 | 5% Pd/C | 5% Pd on Carbon | Johnson | |
| | A503023-5 | (54.36% H₂O) | Matthey | |
| | | | Catalysts | |
| 11 | 5% Pd/C | 5% Pd on Carbon | Johnson | |
| | A503032-5 | (65.72% H₂O) | Matthey | |
| | | | Catalysts | |
| 12 | 5% Pd/C | 5% Pd on Carbon | Johnson | |
| | A503038-5 | (63.41% H₂O) | Matthey | |
| | | | Catalysts | |
| 13 | 5% Pd/C | 5% Pd on Carbon | Johnson | |
| | A102023-5 | (55.98% H₂O) | Matthey | |
| | | | Catalysts | |
| 14 | 5% Pd/C | 5% Pd on Carbon | Johnson | |
| | A102038-5 | (64.57% H₂O) | Matthey | |
| | | | Catalysts | |
| 15 | 5% Pd (S)/C | 5% Pd on Carbon, Sulfided | Johnson | |
| | A103038-5 | (59.74% H₂O) | Matthey | |
| | | | Catalysts | |
| 16 | 5% Pd/Al₂O₃ | 5% Pd on alumina | Johnson | |
| | A302011-5 | (0.46% H₂O) | Matthey | |
| | | | Catalysts | |
| 17 | 5% Pd/Al₂O₃ | 5% Pd on alumina | Johnson | |
| | A302099-5 | (0.52% H₂O) | Matthey | |
| | | | Catalysts | |
| 18 | 5% Pd/CaCO₃ | 5% Pd on calcium carbonate | Johnson | |
| | A302060-5 | (0.73% H₂O) | Matthey | |
| | | | Catalysts | |
| 19 | 5% | 5% Pd on calcium carbonate | Johnson | |
| | Pd(Pb)/CaCO₃ | with lead | Matthey | |
| | A305060-5 | (0.69% H₂O) | Catalysts | |
| 20 | 5% | 5% Pd on calcium carbonate | Johnson | |
| | Pd(Pb)/CaCO₃ | with lead | Matthey | |
| | A306060-5 | (0.72% H₂O) | Catalysts | |
| 21 | 5% Pd/BaSO₄ | 5% Pd on barium sulfate | Johnson | |
| | A308053-5 | (0.75% H₂O) | Matthey | |
| | | | Catalysts | |
| 22 | 4% Pd-1% Pt/C | 4% Pd & 1% Pt on carbon | Johnson | |
| | E101049-4/1 | (54.30% H₂O) | Matthey | |
| | | | Catalysts | |
| 23 | 4% Pd-1% Pt/C | 4% Pd & 1% Pt on carbon | Johnson | |
| | E101023-4/1 | (55.88% H₂O) | Matthey | |
| | | | Catalysts | |
| 24 | 4.5% Pd-0.5% | 4.5% Pd & 0.5% Rh on carbon | Johnson | |
| | Rh/C | (61.48% H₂O) | Matthey | |
| | F101032-4.5/0.5 | | Catalysts | |
| 25 | 4.5% Pd- | 4.5% Pd & 0.5% Rh on | Johnson | |
| | 0.5% Rh/C | Carbon | Matthey | |
| | F101038-4.5/0.5 | (52.51% H₂O) | Catalysts | |
| 26 | 3% Pt/C | 3% platinum on carbon | Johnson | |
| | B103032-3 | (67.93% H₂O) | Matthey | |
| | | | Catalysts | |
| 27 | 5% Pt/C | 5% platinum on carbon | Johnson | |
| | B103032-5 | (59.45% H₂O) | Matthey | |
| | | | Catalysts | |
| 28 | 5% Pt/C | 5% platinum on carbon | Johnson | |
| | B103018-5 | (55.90% H₂O) | Matthey | |
| | | | Catalysts | |
| 29 | 5% Pt/C | 5% platinum on carbon | Johnson | |
| | B102022-5 | (46.67% H₂O) | Matthey | |
| | | | Catalysts | |
| 30 | 5% Pt/C | 5% platinum on carbon | Johnson | |
| | B104032-5 | (62.06% H₂O) | Matthey | |
| | | | Catalysts | |
| 31 | 5% Pt/C | 5% platinum on carbon | Johnson | |
| | B501032-5 | (67.49% H2O) | Matthey | |
| | | | Catalysts | |
| 32 | 5% Pt/C | 5% platinum on carbon | Johnson | |
| | B501018-5 | (54.01% H₂O) | Matthey | |
| | | | Catalysts | |
| 33 | 5% Pt/(Bi)/C | 5% platinum& Bismuth 5% on | Johnson | |
| | B503032-5 | carbon | Matthey | |
| | | (59.40% H₂O) | Catalysts | |
| 34 | 5% Pt/(S)/C | 5% platinum on carbon. Sulfide | Johnson | |
| | B109032-5 | wet | Matthey | |
| | | (60.68% H₂O) | Catalysts | |
| 35 | 5% Pt/(S)/C | 5% platinum on carbon. Sulfide | Johnson | |
| | B106032-5 | wet | Matthey | |
| | | (62.09% H₂O) | Catalysts | |
| 36 | 5% Pt/Al2O3 | 5% platinum on alumina | Johnson | |
| | B301013-5 | (2.84% H₂O) | Matthey | |
| | | | Catalysts | |
| 37 | 5% Pt/Al₂O₃ | 5% platinum on alumina | Johnson | |
| | B301099-5 | (3.28% H₂O) | Matthey | |
| | | | Catalysts | |
| 38 | 5% Rh/C | 5% rhodium on carbon | Johnson | |
| | C101023-5 | (47.61% H₂O) | Matthey | |
| | | | Catalysts | |
| 39 | 5% Rh/C | 5% rhodium on carbon | Johnson | |
| | C101038-5 | (64.21% H₂O) | Matthey | |
| | | | Catalysts | |
| 40 | 5% Rh/Al₂O₃ | 5% rhodium on alumina | Johnson | |
| | C301011-5 | (4.74% H₂O) | Matthey | |
| | | | Catalysts | |
| 41 | 5% Ru/C | 5% ruthenium on carbon | Johnson | |
| | D101023-5 | (62.59% H₂O) | Matthey | |
| | | | Catalysts | |
| 42 | 5% Ru/C | 5% ruthenium on carbon | Johnson | |
| | C101002-5 | (58.46% H₂O) | Matthey | |
| | | | Catalysts | |
| 43 | 5% Rh/Al₂O₃ | 5% ruthenium on alumina | Johnson | |
| | D302011-5 | (0.99% H₂O) | Matthey | |
| | | | Catalysts | |
| 44 | 5% Ru- | 5% ruthenium % 0.25 | Johnson | |
| | 0.25% Pd/C | palladium on carbon | Matthey | |
| | C101038-5 | (53.15% H₂O) | Catalysts | |
| 45 | F105N/W 5% | 5% Pt on activated Carbon | Evonik | |
| | | (55% H₂O) | | |
| 46 | F1082 QHA/W | 5% Pt on activated Carbon | Evonik | |
| | 3% | (63.5% H₂O) | | |
| 47 | F1015 RE/W | 5% Pt on activated Carbon | Evonik | |
| | 5% | (62.3% H₂O) | | |
| 48 | CF 1082 BV/W | 1% Pt + 2% Vanadium on | Evonik | |
| | 1% Pt + 2% V | activated carbon (61.5% H₂O) | | |
| 49 | G106 N/W 5% | 5% Rh on activated Carbon | Evonik | |
| | | (65.4% H₂O) | | |
| 50 | H198 P/W | 5% Ru on activated Carbon | Evonik | |
| | 5% Ru % | (58.7% H₂O) | | |
| 51 | Noblyst P1093 | 5% Palladium on activated | Evonik | |
| | 5% | Carbon (55.5% H₂O) | | |
| 52 | Noblyst P1070 | 10% Palladium on activated | Evonik | |
| | 5% | Carbon (53.5% H₂O) | | |
| 53 | Noblyst P1092 | 5% Palladium on activated | Evonik | |
| | 5% | Carbon (55.5% H₂O) | | |
| 54 | Noblyst P1109 | 5% Palladium on activated | Evonik | |
| | 5% | Carbon (56.6% H₂O) | | |
| 55 | Noblyst P1090 | 5% Palladium on activated | Evonik | |
| | 5% | Carbon (53.5% H₂O) | | |
| 56 | Noblyst P1086 | 5% Palladium on activated | Evonik | |
| | 5% | Carbon (55% H₂O) | | |
| 57 | 46-1710 CAS# | 0.6% Palladium on activated | | |
| | 7440-05-3 | Carbon, unreduced (50% H₂O | | |
| | | wet paste) | | |
| 58 | 46-1901 CAS# | 5% Palladium on activated peat | | |
| | 7440-05-3 | Carbon, unreduced (50% H₂O | | |
| | | wet paste) | | |
| 59 | 46-1902 CAS# | 5% Palladium on activated | | |
| | 7440-05-3 | wood Carbon, reduced, dry | | |
| 60 | 46-1903 CAS# | 5% Palladium on activated | | |
| | 7440-05-3 | wood Carbon, reduced, 50% | | |
| | | water wet paste | | |
| 61 | 46-1904 CAS# | 5% Palladium on activated | | |
| | 7440-05-3 | wood Carbon, unreduced (50% | | |
| | | H₂O wet paste) | | |
| 62 | 46-1905 CAS# | 10% Palladium on activated | | |
| | 7440-05-3 | wood Carbon, reduced (50% | | |
| | | H₂O wet paste) | | |
| 63 | 46-1951 CAS# | 5% Palladium on alumina | | |
| | 7440-05-3 | Al₂O₃. reduced | | |
| 64 | 46-1707 CAS# | 20% Palladium on activated | | |
| | 7440-05-3 | carbon Pearlman's catalyst, | | |
| | | unreduced, 50% water wet | | |
| | | paste) | | |
| 65 | 78-1611 CAS# | 5% Platinum on activated wood | | |
| | 7440-06-4 | carbon, reduced, dry | | |
| 66 | 78-1612 CAS# | 5% Platinum on activated wood | | |
| | 7440-06-4 | carbon, reduced, 50% H₂O wet | | |
| | | paste | | |
| 67 | 78-1613 CAS# | 5% Platinum on activated wood | | |
| | 7440-06-4 | carbon, unreduced, 50% H₂O | | |
| | | wet paste | | |
| 67 | 44-4065 CAS# | 5% Ruthenium on activated | | |
| | 7440-18-8 | carbon, reduced, 50% H₂O | | |
| | | wet paste | | |
| 68 | 45-1875 CAS# | 5% Rhodium on activated wood | | |
| | 7440-16-6 | carbon, reduced, 50% H₂O wet | | |
| | | paste | | |

Temperature, solvent, catalyst, reactor configuration, pressure, amount of added hydrogen gas, catalyst concentration, metal loading, catalyst support, starting feed, additives and mixing rate are all parameters that can affect the conversions described herein. The relationships among these parameters may be adjusted to effect the desired conversion, reaction rate, and selectivity in the reaction of the process.

In some embodiments, the methods provided herein are performed at temperatures from about 25° C. to about 350° C. For example, the methods can be performed at a temperature of at least about 100° C. In some embodiments, a method provided herein is performed at a temperature of about 100° C. to about 200° C. For example, a method can be performed at a temperature of about 150° C. to about 180° C.

The methods described herein may be performed neat, in water or in the presence of an organic solvent.

In some embodiments, the reaction solvent comprises water. Exemplary organic solvents include hydrocarbons, ethers, and alcohols. In some embodiments, alcohols can be used, for example, lower alkanols, such as methanol and ethanol. The reaction solvent can also be a mixture of two or more solvents. For example, the solvent can be a mixture of water and an alcohol.

The methods provided herein can be performed under inert atmosphere (e.g., N₂ and Ar). In some embodiments, the methods provided herein are performed under hydrogen or, nitrogen or mixture of nitrogen and hydrogen. For example, the methods can be performed under a hydrogen pressure of about 20 psi to about 1000 psi. In some embodiments, a method as described herein is performed under a hydrogen pressure of about 200 psi and 450 psi.

In some embodiments, additional reactants can be added to the methods described herein. For example, a base such as NaOH can be added to the reaction.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), gas chromatography (GCMS, GCFID) or thin layer chromatography (TLC). Compounds can be purified by those skilled in the art by a variety of methods, including high performance liquid chromatography (HPLC) (“Preparative LC-MS Purification: Improved Compound Specific Method Optimization” K. F. Blom, et al., J. Combi. Chem. 6(6) (2004), which is incorporated herein by reference in its entirety) and normal phase silica chromatography.

### DEFINITIONS

It is appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about”, whether or not the term is explicitly used, unless explicitly stated otherwise. As used herein, the singular forms “a,” “an,” and “the” include plural referents unless the context clearly dictates otherwise.

The term “salt” includes any ionic form of a compound and one or more counter-ionic species (cations and/or anions). Salts also include zwitterionic compounds (i.e., a molecule containing one more cationic and anionic species, e.g., zwitterionic amino acids). Counter ions present in a salt can include any cationic, anionic, or zwitterionic species. Exemplary anions include, but are not limited to: chloride, bromide, iodide, nitrate, sulfate, bisulfate, sulfite, bisulfite, phosphate, acid phosphate, perchlorate, chlorate, chlorite, hypochlorite, periodate, iodate, iodite, hypoiodite, carbonate, bicarbonate, isonicotinate, acetate, trichloroacetate, trifluoroacetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, trifluormethansulfonate, ethanesulfonate, benzensulfonate, p-toluenesulfonate, p-trifluoromethylbenzenesulfonate, hydroxide, aluminates and borates. Exemplary cations include, but are not limited to: monovalent alkali metal cations, such as lithium, sodium, potassium, and cesium, and divalent alkaline earth metals, such as beryllium, magnesium, calcium, strontium, and barium. Also included are transition metal cations, such as gold, silver, copper and zinc, as well as non-metal cations, such as ammonium salts.

An “ester” as used herein includes, as nonlimiting examples, methyl esters, ethyl esters, and isopropyl esters, and esters which result from the addition of a protecting group on a corresponding carboxyl moiety.

A “lactone” as used herein refers to the cyclic ester compounds which result from the condensation of an alcohol group and a carboxylic acid group on the compounds provided herein. A nonlimiting example is the lactone which results from the condensation of homocitric acid, or its salts (ie. homocitric acid lactone).

As used herein, chemical structures which contain one or more stereocenters depicted with bold and dashed bonds (i.e., [symbol] ) are meant to indicate absolute stereochemistry of the stereocenter(s) present in the chemical structure. As used herein, bonds symbolized by a simple line do not indicate a stereo-preference. Unless otherwise indicated to the contrary, chemical structures, which include one or more stereocenters, illustrated herein without indicating absolute or relative stereochemistry encompass all possible steroisomeric forms of the compound (e.g., diastereomers, enantiomers) and mixtures thereof. Structures with a single bold or dashed line, and at least one additional simple line, encompass a single enantiomeric series of all possible diastereomers.

Compounds, as described herein, can also include all isotopes of atoms occurring in the intermediates or final compounds. Isotopes include those atoms having the same atomic number but different mass numbers. For example, isotopes of hydrogen include tritium and deuterium.

The term, “compound,” as used herein is meant to include all stereoisomers, geometric isomers, tautomers, and isotopes of the structures depicted. Compounds herein identified by name or structure as one particular tautomeric form are intended to include other tautomeric forms unless otherwise specified.

All compounds, salts, esters, and lactones thereof, can be found together with other substances such as water and solvents (e.g. hydrates and solvates).

In some embodiments, the compounds described herein, or salts, esters, or lactones thereof, are substantially isolated. By “substantially isolated” is meant that the compound is at least partially or substantially separated from the environment in which it was formed or detected. Partial separation can include, for example, a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof. Methods for isolating compounds and their salts are routine in the art.

### EXAMPLES

### Example 1—Testing of Palladium Catalysts

A number of palladium catalysts were tested to optimize reaction conditions for converting homocitric acid lactone to 1,2,4-butanetricarboxylic acid. The catalysts are referred to using the numerical index as shown in Table 1. For example, catalyst No. 6 is a 5% Pd/C from Johnson Matthey containing 56% water. Experiments were performed using Pd-based catalysts supported on carbon with different water amounts. Initially, 1 ml of a 0.25 M homocitric acid lactone solution in dry methanol was used and the catalyst loading was 0.5 mol % (calculated on dry powder basis). The reaction time was 16 hours in all cases under 200 psi of H₂. The reaction products were analyzed using GC/MS (Agilent, 5975B, inert, XL, EI/CI). The evaluation of the catalysts is based on qualitative results of the GC/MS data.

### Materials and Methods

Activation Temperature/Method

Three different types of activation procedure/methods were used. Methods A and B were performed under H₂ pressure (200 psi) while method C was performed using H₂ flow. For the purposes of comparison, catalyst No 59, that was already dry and reduced as received, was also tested.

Method A: Activation at 100° C. Under H₂ Pressure

The desired amount of supported catalyst was transferred to the HP reactor (Symyx Discovery Tools) and the following steps were performed for its activation,

a. Annealing at 100° C. under 400 psi of N₂ for 1 hour

b. Annealing at 100° C. under 200 psi of H₂ for 2 hours

This temperature (100° C.) was selected since it is the lowest activation temperature recommended for Pd-based catalysts according BASF and JM.

Method B: Activation at 180° C. Under H₂ Pressure

The desired amount of supported catalyst was transferred in the HP reactor (Symyx Discovery Tools) and the following steps were performed for its activation:

a. Annealing at 140° C. under 400 psi of N₂ for 1 hour,

b. Annealing at 180° C. under 200 psi of H₂ for 2 hours.

The temperature of 180° C. is the maximum temperature that can be achieved with the HPR at the High Throughput facility.

Method C: Activation at 180° C. Under H₂ Flow

Two Pd/C supported catalysts, namely Nos. 7 and 51 were transferred to a quartz reactor and the following steps were followed for its activation:

a. Step-by step annealing up to 400° C. under flow of Ar

b. Step-by step annealing up to 400° C. under flow of H₂

The activation of supported catalysts is usually performed at high temperature e.g. T>200° C. initially under flow of an inert gas and then under flow of H₂. As described herein, the activation was performed using initially low contents of H₂ to avoid exotherms and was gradually increased so as to achieve the reduction of Pd.

Reaction Temperature

The lactone hydrogenolysis reaction was performed under 200 psi of H₂ at two different temperatures: 100 and 180° C. for 16 hours. Catalysts activated under different conditions were tested in order to find the best combination of activation temperature/method and reaction temperature.

Effect of pH

The effect of base, NaOH on the reaction mixture was also evaluated. For these experiments two different catalysts were chosen (No. 6 and No. 59) and to the reaction mixture were added 1, 2 and 3 equiv. of NaOH. The reaction was performed at 100° C. under 200 psi of H₂.

### Results and Discussion

Table 2 summarizes the catalysts that were tested

TABLE 2
Summary of the catalysts using the numerical index of Table 1.

| Activation/Reaction | | |
|---|---|---|
| Temperature | 100° C. | 180° C. |
| Method A | 7, 9, 13, | 7, 51 |
| | 51, 53, 54 | |
| Method B | 51 | 7, 9, 13, |
| | | 51, 53, 54 |
| Method C | 7, 51 | 7, 51 |
| Commercial dry and | 59 | 59 |
| reduced | | |

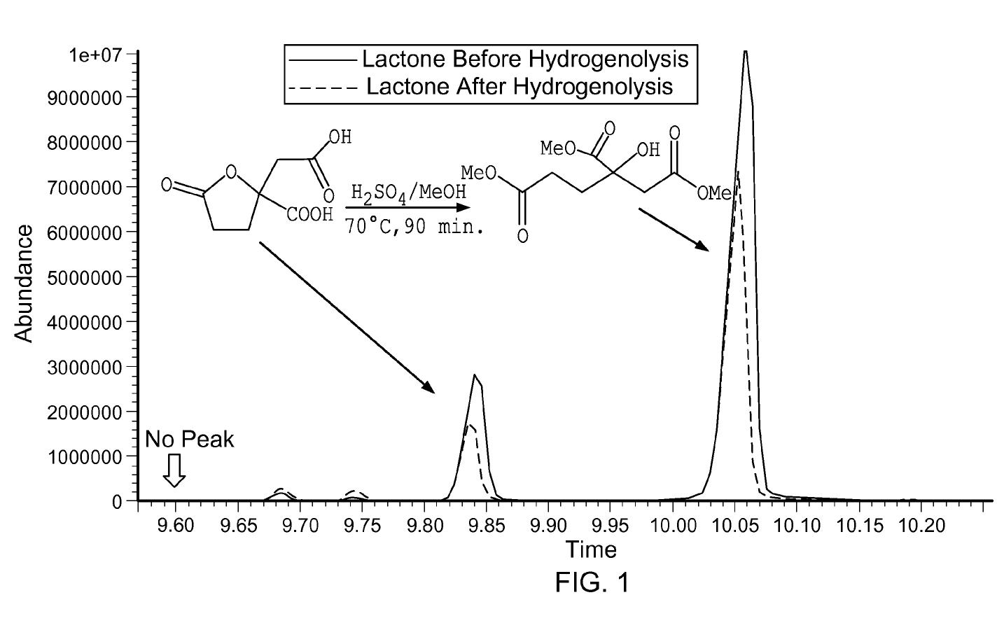

A control sample of homoctiric acid lactone was prepared in the same manner as the test samples but without the addition of catalyst. FIG. 1 provides the GC/MS chromatogram of the control. Two peaks of high intensity were detected at around 10.05 and 9.82 minutes. These peaks are characteristic of the starting material.

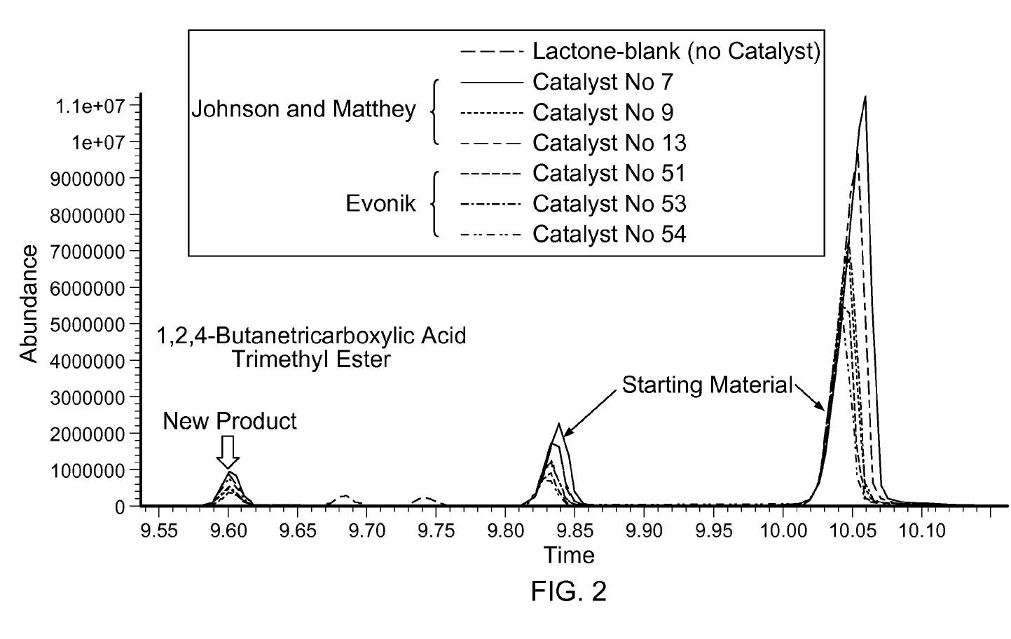

Preliminary studies were performed using Pd/C catalysts, activated at 100° C. (Method A) and at relatively low reaction temperature (100° C.). For these experiments, six different catalysts were tested and the chromatograms of the final product after the reaction are shown in FIG. 2.

FIG. 2 shows that all of the catalysts tested were active for hydrogenolysis of homocitric acid lactone. However, there were no significant differences between the catalysts in terms of product distribution. A new peak was detected at 9.6 minutes that is attributed to the product 1,2,4-butanetricarboxylic acid based on GC-mass spectrometry and NIST library. As was detected in control lactone sample, with all these various catalysts as well, the existence of the two peaks at 9.82 and at 10.05 min reveal that a significant amount of the starting materials have not reacted under the specified reaction conditions. At these conversions, differences in catalyst activity as a function of carbon support were not discernible.

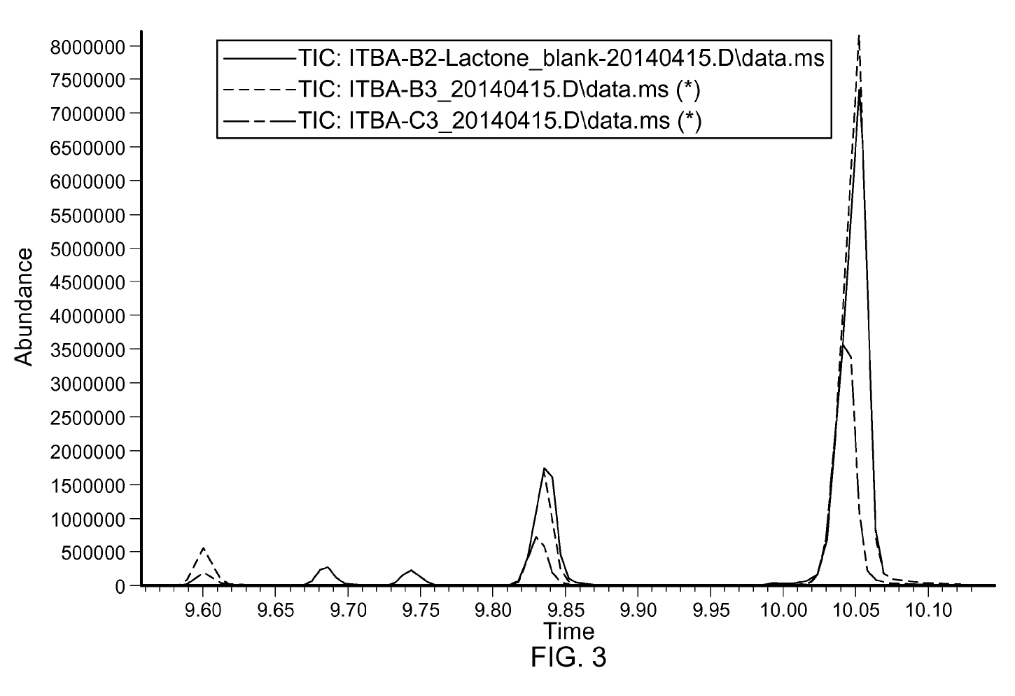

The catalysts at also activated at higher temperature and, more specifically, at 180° C. Thus, catalyst No 6 was activated according to Method B (at 180° C.) and the reaction was performed at 100° C. Moreover, for the purpose of comparison the dry and reduced Pd/C catalyst was also tested. The obtained chromatograms are presented in FIG. 3.

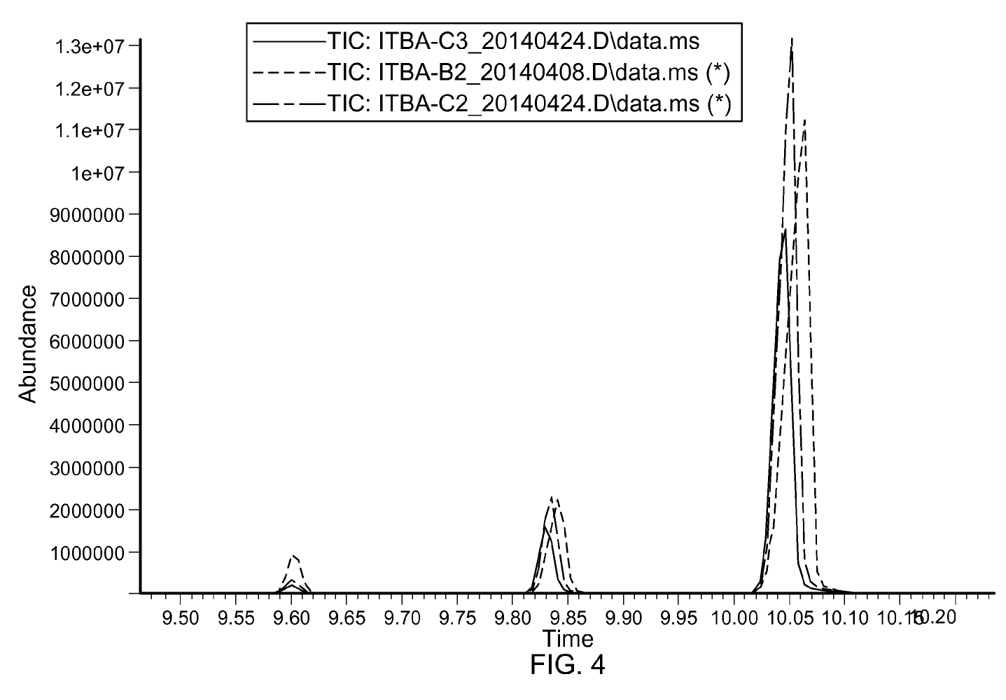

As shown in FIG. 3, most of the starting material did not react even in the case of catalyst 59 (commercially reduced and dried catalyst). This is further supported by the chromatograms of the final product after reaction at 100° C. using catalyst No. 51 activated with all three different activation methods (FIG. 4). This indicates that the hydrogenolysis reaction must be performed at higher temperatures.

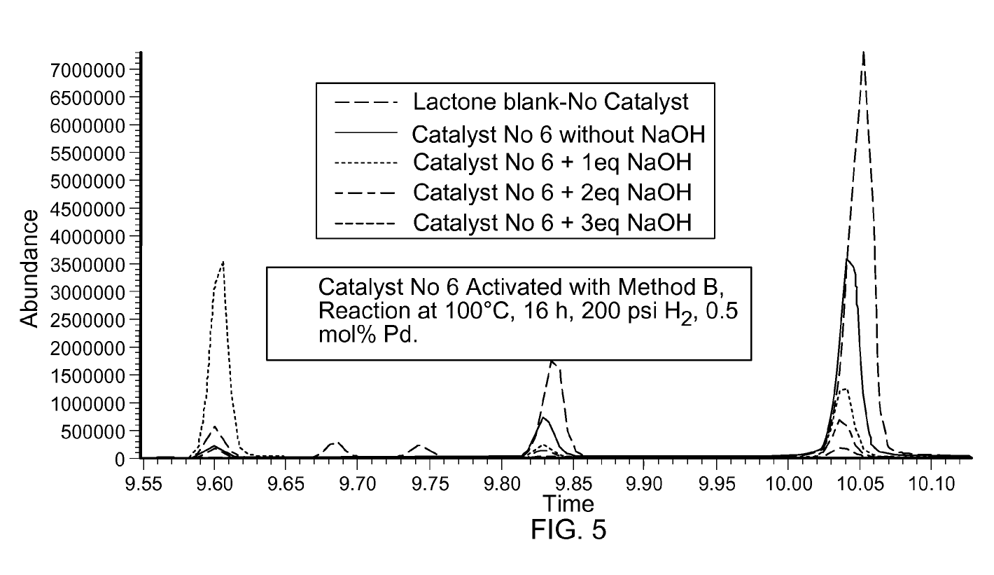

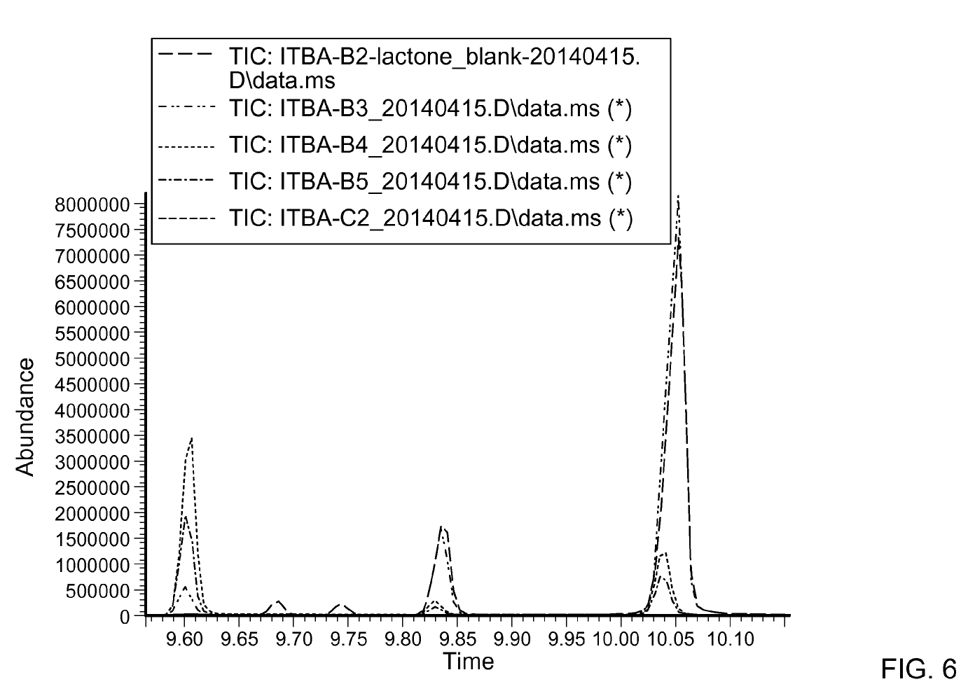

Further experiments were performed in order to investigate the influence of NaOH on the reaction. Experiments were performed using catalyst No. 6 activated following method B (180° C.) and also the already dry and reduced commercial catalyst No. 59. The reaction was performed at 100° C. for 16 hours. GC/MS chromatograms obtained before and after the addition of 1, 2, and 3 equivalents of NaOH are presented in FIGS. 5 and 6.

Addition of 1 equivalent of NaOH in both cases causes the increase in the intensity of the peaks at around 9.60 minutes whereas a decrease in the intensity of the peaks were observed at 9.82 and 10.05 min compared to the control lactone. The fact that these two peaks (at 9.82 and 10.05 minutes) were still detected and are of relatively high intensity (blue line) with low intensity pick at 9.6 minutes without the addition of NaOH implies that the addition of a relatively small amount of NaOH (1 equiv.) appears to facilitates an increase of the conversion of the starting material under the specified reaction conditions. The addition of 2 or 3 equivalents of NaOH, on the other hand, dried up the reaction aliquots significantly during/after the reaction. As the total reaction volume is only 1 ml, the drying effect could account for small amount of product formation observed using 3 equivalents of NaOH.

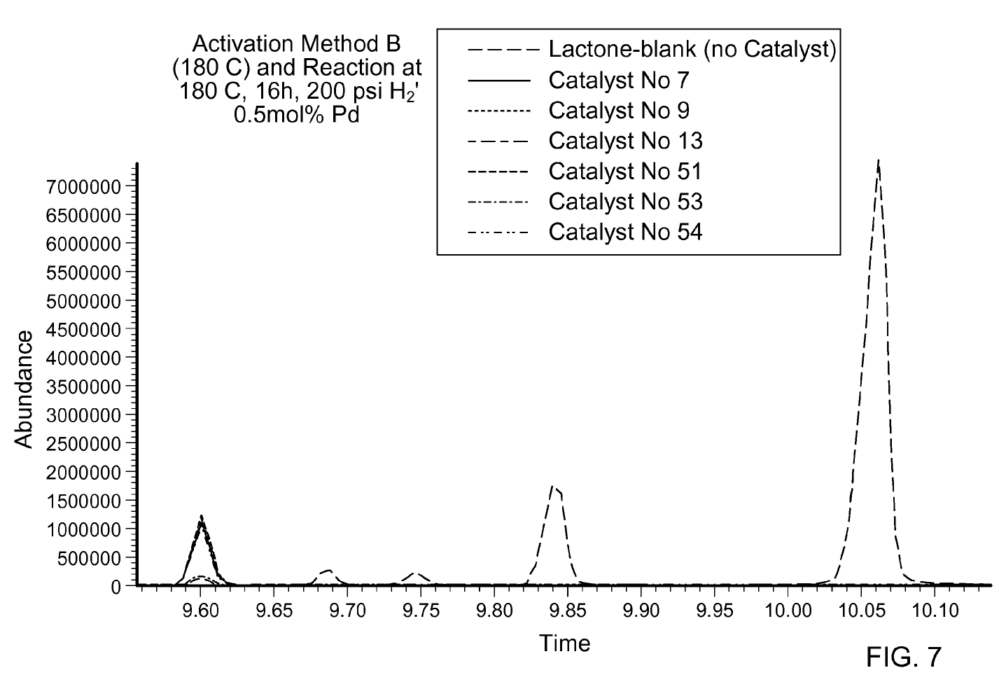

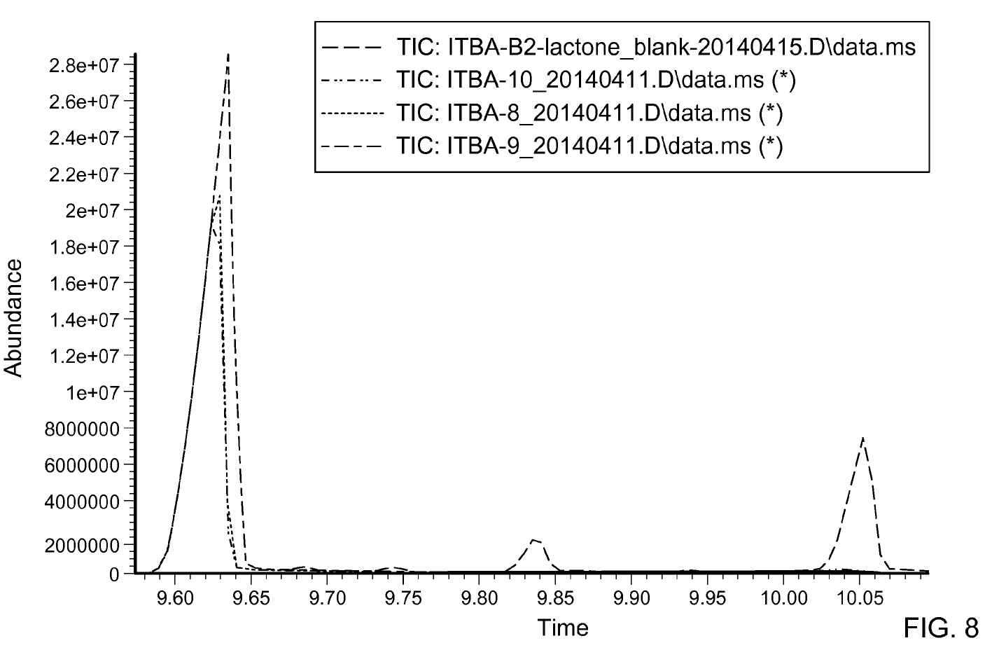

To investigate whether the increase of the reaction temperature can cause higher conversions of homocitric acid lactone, the following steps were performed at higher reaction temperatures. The same catalysts tested previously (at 100° C., FIG. 1) were activated at 180° C. using method B and were added to the homocitrate/methanol reaction solution. The reaction was performed at 180° C. under 200 psi of H₂ for 16 h. The obtained chromatograms are presented in FIG. 7 in comparison with a blank sample (lactone without catalyst). In all cases, the obtained chromatograms indicated that conversion to the desired product is quantitative. Additionally, the performance of catalysts Nos. 7 and 51 reduced at 400° C. under H₂ flow and, for comparison, the reduced and dry catalyst No. 59 were also tested at 180° C. (reaction temperature. The chromatograms of the final products are shown in FIG. 8; complete conversion of homocitric acid lactone was achieved in all cases.

### Example 2—Reaction Optimization

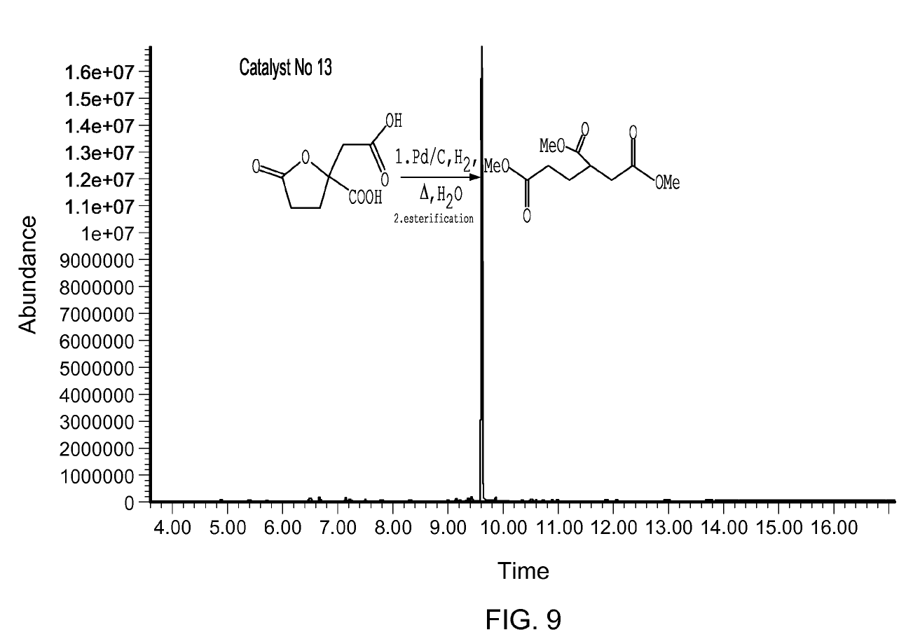

Conversion of homocitric acid lactone (0.25 mmol) to 1,2,4-butanetricarboxylic acid was tested at a lower temperature of 150° C. for 4 hours using catalyst No. 13 (0.5 mol % Pd(5% Pd/C)) in water. As shown in FIG. 9, conversion to the product was quantitative.

### Example 3—Conversion of Homocitric Acid Lactone to Adipic Acid

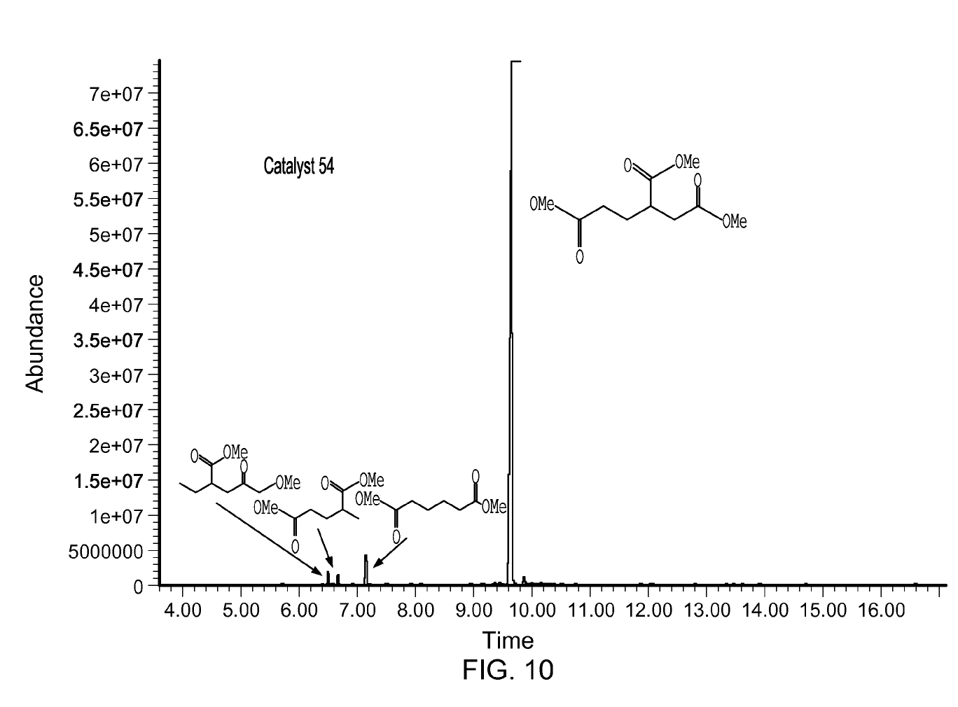

By optimizing catalyst concentration and using the general reaction conditions provided in Example 2, it was observed that conversion of homocitric acid lactone to adipic acid, 2-ethylsuccinic acid, and 2-methylpentanedioic acid occurred in a single pot reaction. Specifically, reactions with catalyst Nos. 6, 12, and 54 exhibited quantitative conversion of the lactone to the tricarboxylic acid and further underwent selective decarboxylation to produce three product peaks. FIG. 10 provides an exemplary chromatogram. Increasing the reaction temperature to 180° C. did not appear to have a significant effect on the decarboxylation products observed.

### Example 4—Pd(S)/C Catalyst

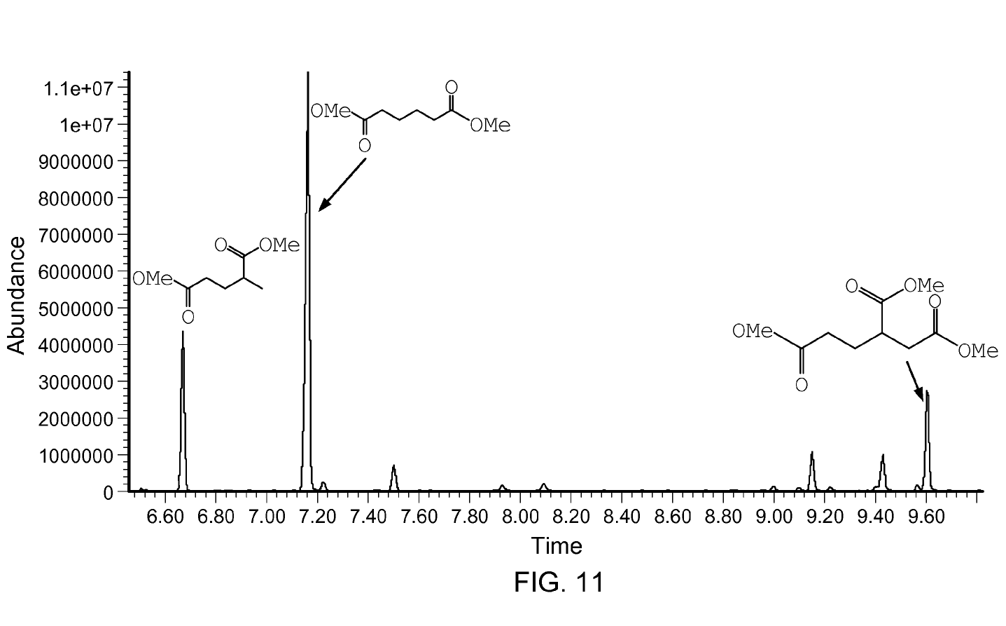

As shown in FIG. 11, combining homocitric acid lactone as described above with catalyst No. 15 (a Pd(S)/C catalyst) and water at 180° C. for 16 hours under 200 psi H₂ resulted in significant production of decarboxylation products. Increasing the residence time to 22 hours did not have a significant effect on the yield of adipic acid (data not shown). Addition of 0.5 equivalents of base also did not improve conversion of lactone to adipic acid, but it did increase the production of 2-ethylsuccinic acid.

### Example 5—Comparison of Pt(S)/C Catalyst with Pt/C Supported Catalyst

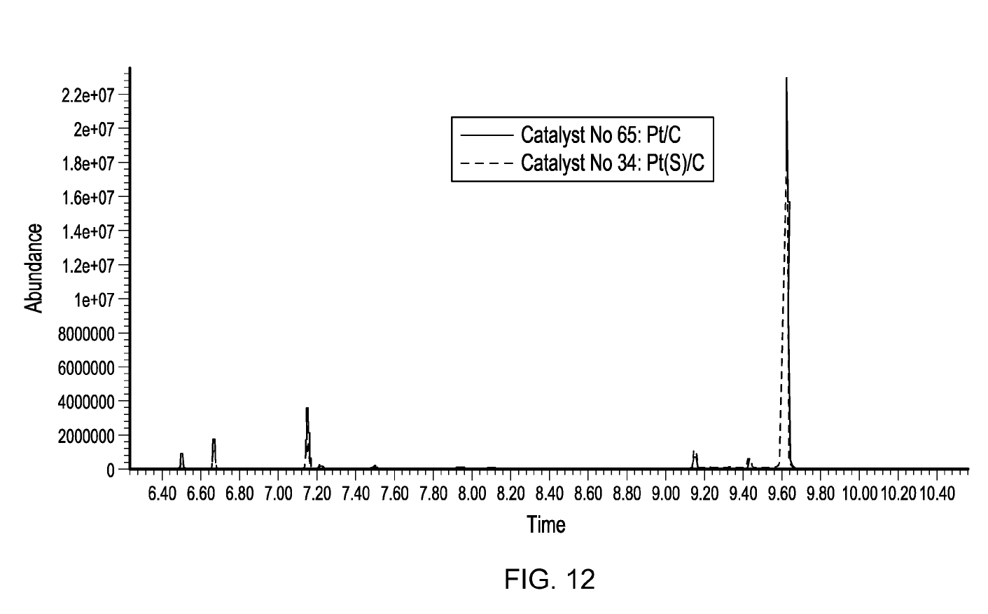

As shown in FIG. 12, a reaction of homocitric acid lactone at 150° C. in water under 200 psi H₂ for 42 hours in the presence of 1 mol % Pt (catalyst Nos. 65 and 34), shows some decarboxylation of lactone at lower temperatures, but the reaction appeared to be less selective than the Pd(S)/C catalytic reactions.

### Example 6—Pd/CaCO₃ Catalyst

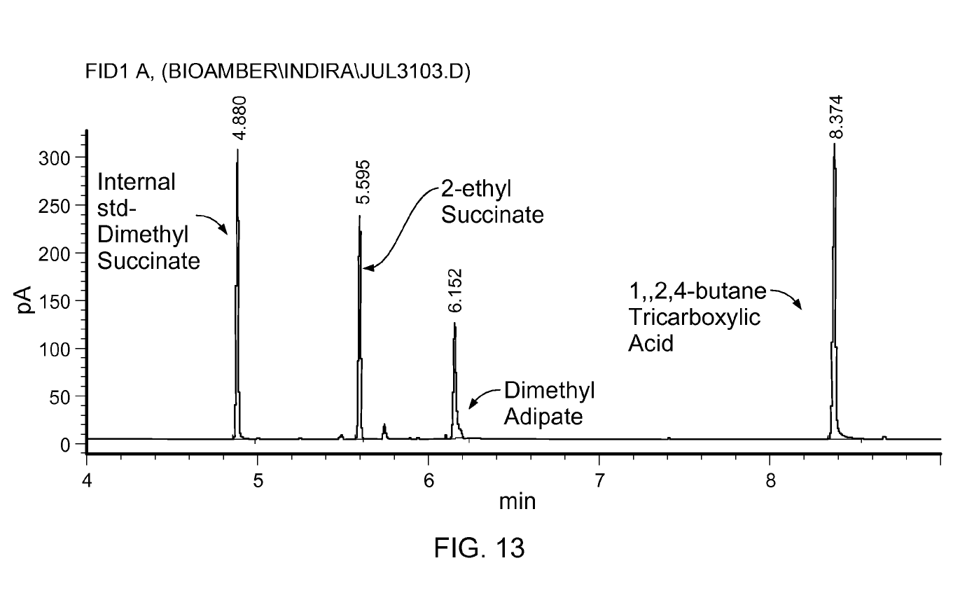

As shown in FIG. 13, combining homocitric acid lactone (0.12 M) as described above with catalyst No. 18 (a Pd/CaCO₃ catalyst, 1 mol % Pd) and water at 180° C. for 16 hours under 450 psi H₂ resulted in significant production of decarboxylation products.

### Example 7—Pd/BaSO₄ Catalyst

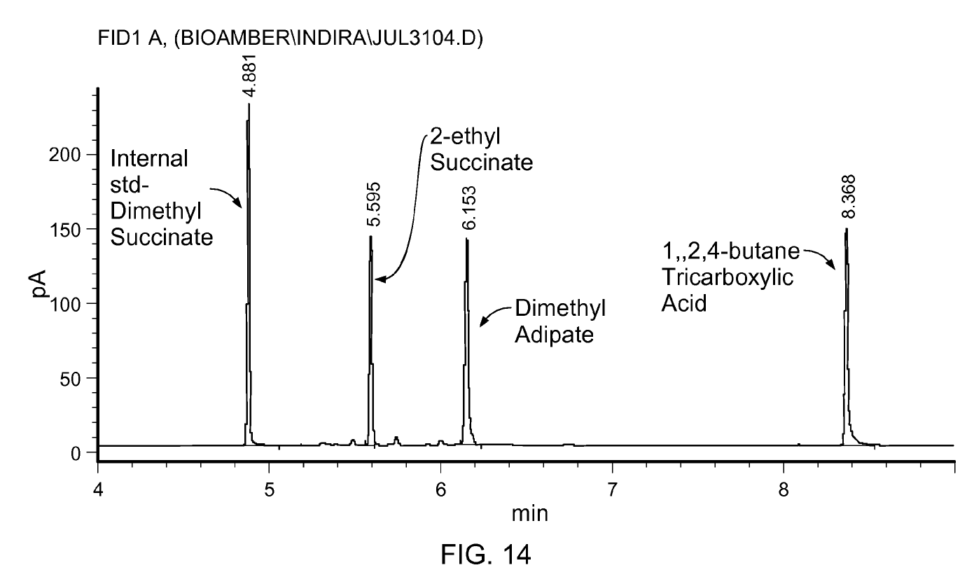

As shown in FIG. 14, combining homocitric acid lactone (0.12 M) as described above with catalyst No. 21 (a Pd/BaSO₄ catalyst, 1 mol % Pd) and water at 180° C. for 16 hours under 450 psi H₂ resulted in significant production of decarboxylation products.

### Example 8—Catalyzed Thermolysis of Lactone with No Added Hydrogen

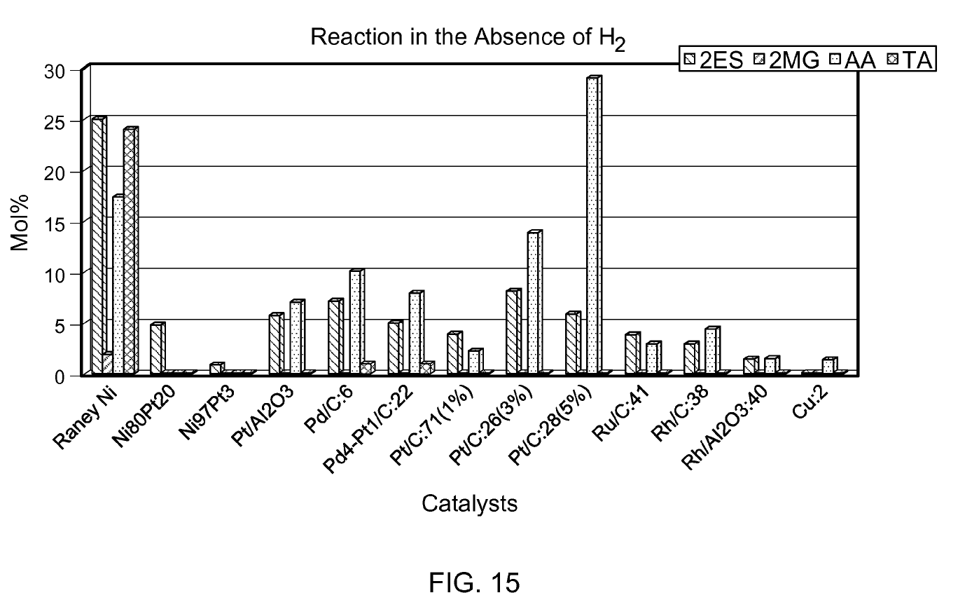

As shown in FIG. 15, combining homocitric acid lactone (0.12 M) as described above with supported metal catalysts (1 mol % metal) and water at 180° C. for 16 hours under 450 psi N₂ resulted in significant production of decarboxylation products. FIG. 15 is the representative example of each of the few supported catalysts tested. 5% Pt/C showed remarkable selectivity to adipic acid in the absence of added H₂ and in the presence of 450 psi of N₂ pressure compared to other Pt/C (1% Pt and 3% Pt on carbon) and Pt/Al₂O₃. Ni-based catalysts favours ethyl succinic acid over adipic acid.

### Example 9—Catalyzed Conversion of Homocitric Acid Lactone to Adipic Acid Under Mixed Gas N₂/H₂ (95:5)

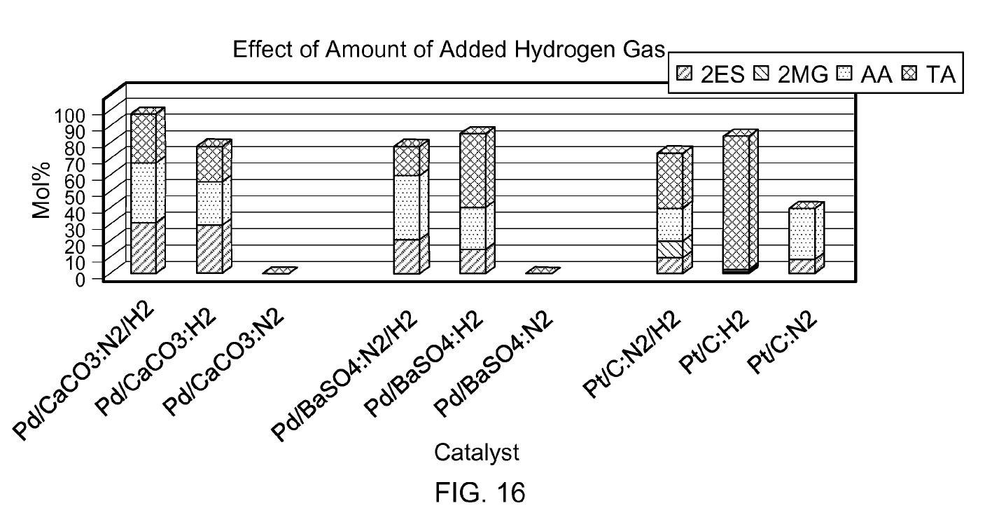

As shown in FIG. 16, combining homocitric acid lactone (0.12 M) as described above with supported metal catalysts (1 mol % metal) and water at 180° C. for 16 hours under 450 psi N₂/H₂ (95:5) mixed gas pressure resulted in significant production of decarboxylation products. Representative examples were presented in FIG. 16. Significant improvement in adipic acid formation was observed with the mixture of H₂/N₂ gas (5:95)% with CaCO₃ and BaSO₄ supported Pd catalysts. Pt/C favours decarboxylation to di-acids with lesser added H₂ under the specified reaction conditions. A comparative example is presented in FIG. 16.

### Example 10—Effect of Sulfur Containing Solvent (DMSO) for the Conversion of Homocitric Acid Lactone to Adipic Acid

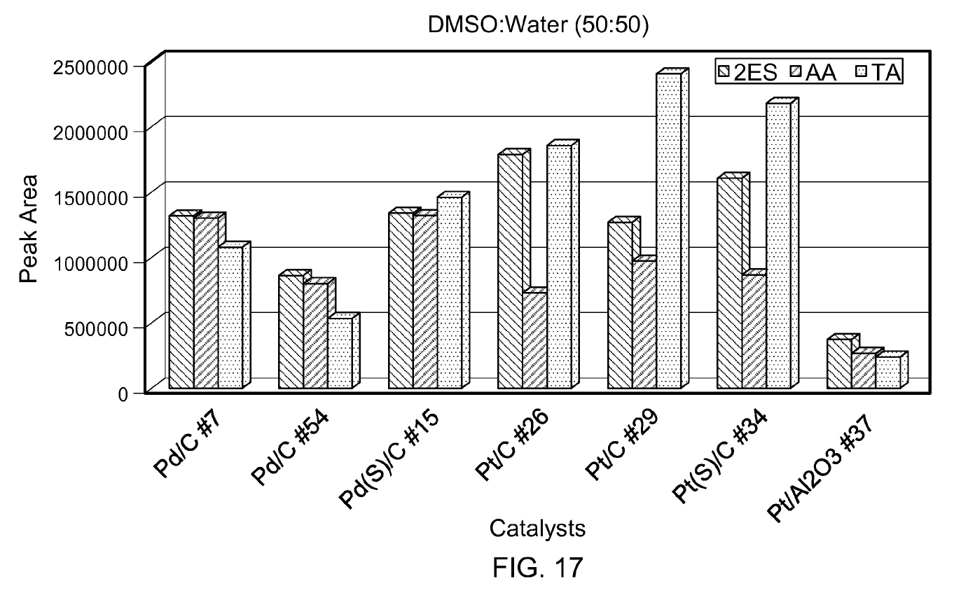

As shown in FIG. 17, combining homocitric acid lactone (0.12 M) as described above with supported metal catalysts (1 mol % metal) and water at 180° C. for 16 hours under 450 psi H₂ resulted in significant production of decarboxylation products. FIG. 17 is the representative example of each of the few supported catalysts tested. Additionally metals with different supports such as Pd/CaCO₃, Pd/BaSO₄, Pt/Al2O₃, Rh, Ru etc were tested as well under the same conditions with 50% mixture of DMSO and water. The catalysts with other supports except carbon showed only traces of diacids formation with sufficient unconverted starting material and intermediate (ethylidene) in presence of DMSO. Qualitative results from GC-MS analysis is presented in the following chart (FIG. 17). Lower DMSO concentration of (10-50%) in water showed enhanced conversion of homocitric aid lactone to adipic acid under the specified reaction conditions. As shown in FIG. 17 Pd and Pt supported on carbon catalysts showed improved activity to adipic acid with DMSO (50% in water). For example catalyst no. 7 Pd/C showed comparable selectivity with sulfided Pd catalyst on carbon in presence of DMSO.

### Example 11—Scale Up Reaction for the Conversion of Homocitric Acid Lactone to Adipic Acid

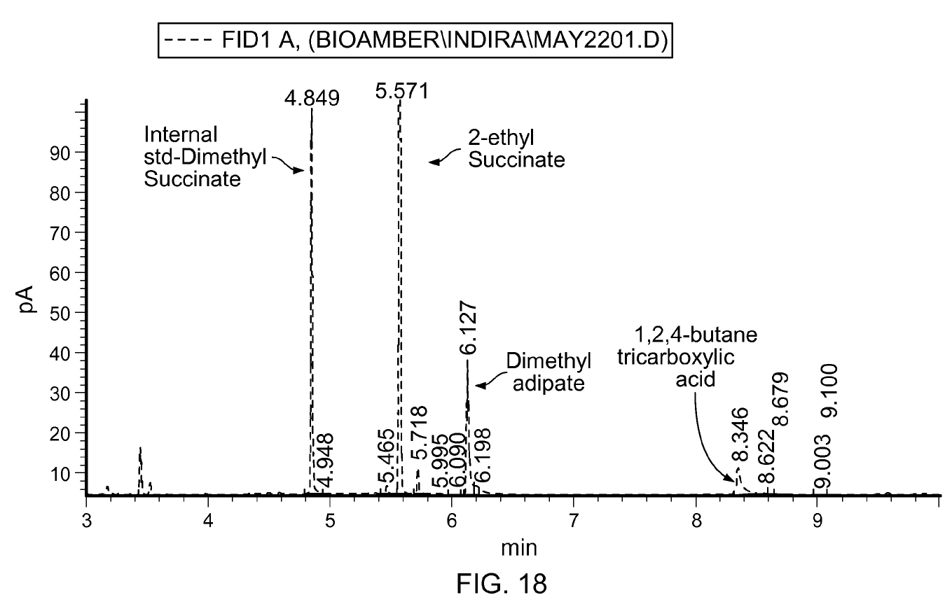

A scale up reaction was performed in a 300 mL autoclave (Parker Autoclave Bolted Closure). As shown in FIG. 18, combining homocitric acid lactone (0.12 M) in presence of internal standard as described above with Pd/CaCO₃ supported catalyst (1 mol % Pd) and water (50 mL) at 200° C. for 16 hours under 500 psi H₂ resulted in significant production of decarboxylation products. Further reaction optimization at larger scale under various reaction parameters (Temperature, pressure, Time, reaction feed, catalyst concentration and supports) in an autoclave can be performed to improve activity and selectivity of adipic acid.

### Example 12—Production of Homocitrate Lactone from Acidophilic Yeast

The following acidophilic yeast that results from this Example 12, can be used to produce homocitrate at greater than 40 g/L. The fermentation broth will have a pH of less than or equal to 3. Therefore, the majority of the homocitrate will be in the lactone form. Thus, it will be easily separated from the fermentation broth and ready for reaction with a catalyst to produce the organic acids described herein.

In some embodiments it may be necessary to knock out URA3, PDC, ALD9091, and GPD1 genes individually or in combinations. The URA3 knockout is necessary in order to facilitate positive and negative selections via the presence or absence of the URA3 gene product when used in combination with genetic manipulations as described below. PCD, ALD9091, and GPD1 are mutations that thought to reduce potential byproducts, namely ethanol and glycerol, and potential increase product yields. Additionally, downstream genes and regulatory genes coding for enzymes in the native yeast pathway maybe modified by up regulation, down regulation, mutation or deletion using a process similar to the gene modification method described below. These genes include the I. orientalis genes that are homologous to the S. cerevisiae for ACO1 (homocitrate dehydratase), ACO2 (homocitrate dehydratase), LYS4 (homoaconitase), LYS12 (homoisocitrate dehydrogenase), LYS2 (alpha aminoadipate reductase), LYS9 (saccharopine dehydrogenase), LYS1 (saccharopine dehydrogenase, L-lysine forming). Altering the expression of these genes or their products could help increase homocitrate production by limiting lysine production through the native pathway. In another embodiment, increased expression of homocitrate dehydratase, native or exogenous, can be utilized to convert homocitrate to homoaconitate to be used as an alternative starting feed for the catalytic reaction, either as part of intact pathway within the cell or enzymatically outside of the cell. In addition, known transcriptional regulation genes including the I. orientalis genes that are homologous to the S. cerevisiae genes such as LYS14 and LYS80, which are known to control the yeast lysine pathway, could also be modified by up regulation, down regulation, mutation or deletion using a process similar to the gene modification method described below. These changes could increase homocitrate production and decrease byproduct formation, name lysine or other intermediates in this pathway. In some circumstances, these mutations may result in complete or partial auxotrophy for lysine. Accordingly, in these circumstances fermentation growth and production conditions could be developed using lysine supplementation to overcome such limitation and provide an economically advantageous fermentation system. Alternatively, fully limiting flux to lysine may be accomplished by nitrogen limiting conditions. For example, conditions could be developed for a growth phase where enough nitrogen was supplied as to make enough lysine, but during production nitrogen limitation would only allow the earlier pathway step such as those producing homocitrate to function.

Evolution of an acid tolerant strain (to homocitrate or homocitrate lactone) can be performed. An I. orientalis strain host strain is generated by evolving I. orientalis strain ATCC PTA-6658 for 91 days in a glucose-limited chemostat. The system is fed 15 g/L glucose in a defined medium and operated at a dilution rate of 0.06 h 1 at pH=3 with added homocitrate acid in the feed medium. The conditions are maintained with an oxygen transfer rate of approximately 2 mmol L̂h 1, and dissolved oxygen concentration remains constant at 0% of air saturation. Single colony isolates from the final time point are characterized in two shake flask assays. In the first assay, the isolates are characterized for their ability to ferment glucose to ethanol in the presence of 25 g/L total homocitrate acid with no pH adjustment in the defined medium. In the second assay, the growth rate of the isolates is measured in the presence of 45 g/L of total homocitrate acid, with no pH adjustment in the defined medium. The resulting strain can be termed P-1 it is a single isolate exhibiting the highest glucose consumption rate in the first assay and the highest growth rate in the second assay.

Yeast Base Strain for Cloning

P-2 (a strain based upon strain P-1). Strain P-1 is transformed with linearized integration fragment P2 (having nucleotide sequence SEQ ID NO: 1) designed to disrupt the URA3 gene, using the LiOAc transformation method as described by Gietz et al., in Met. Enzymol. 350:87 (2002). Integration fragment P2 includes a MEL5 selection marker gene. Transformants are selected on yeast nitrogen base (YNB)-melibiose plates and screened by PCR to confirm the integration of the integration piece and deletion of a copy of the URA3 gene. A URA3-deletant strain is grown for several rounds until PCR screening identifies an isolate in which the MEL5 selection marker gene has looped out. The PCR screening is performed using primers having nucleotide sequences SEQ ID NOs: 2 and 3 to confirm the 5′-crossover and primers having nucleotide sequences SEQ ID NOs: 4 and 5 to confirm the 3′ crossover. That isolate is again grown for several rounds on 5-fluoroorotic acid (FOA) plates to identify a strain in which the URA3 marker has looped out. PCR screening is performed on this strain using primers having nucleotide sequences SEQ ID NOs: 2 and 5, identifies an isolate in which both URA3 alleles have been deleted. In a preferred aspect, the strain is selected on 5-fluoroorotic acid (FOA) plates prior to the PCR screening described in the previous sentence. This isolate is named strain P-2.

P-3 (a strain based upon strain P-2). Strain P-2 is transformed with integration fragment P3 (having the nucleotide sequence SEQ ID NO: 6), which is designed to disrupt the PDC gene. Integration fragment P3 contains the following elements, 5′ to 3′: a DNA fragment with homology for integration corresponding to the region immediately upstream of the I. orientalis PDC open reading frame, a PDC transcriptional terminator, the URA3 promoter, the I. orientalis URA3 gene, an additional URA3 promoter direct repeat for marker recycling and a DNA fragment with homology for integration corresponding to the region directly downstream of the I. orientalis PDC open reading frame. A successful integrant (and single-copy PDC deletant) is identified on selection plates lacking uracil and confirmed by PCR using primers having nucleotide sequences SEQ ID NOs: 7 and 8 to confirm the 5′-crossover and primers having nucleotide sequences SEQ ID NOs: 9 and 10 to confirm the 3′-crossover. That integrant is grown for several rounds and plated on 5-fluoroorotic acid (FOA) plates to identify a strain in which the URA3 marker has looped out. The looping out of the URA3 marker is confirmed by PCR. That strain is again transformed with integration fragment P3 to delete the second copy of the native PDC gene. A successful transformant is again identified by selection on selection plates lacking uracil, and further confirmed by culturing the strain over two days and measuring ethanol production. Lack of ethanol production further demonstrates a successful deletion of both copies of the PDC gene in a transformant. That transformant is grown for several rounds and plated on FOA plates until PCR identifies a strain in which the URA3 marker has looped out. The PCR screening is performed using primers having nucleotide sequences SEQ ID NOs: 7 and 8 to confirm the 5′-crossover and SEQ ID NOs: 9 and 10 to confirm the 3′-crossover. That strain is plated on selection plates lacking uracil to confirm the loss of the URA3 marker, and is designated strain P-3.

P-4. Integration fragment P4-1, having nucleotide sequence SEQ ID NO:11, contains the following elements, 5′ to 3′: a DNA fragment with homology for integration corresponding to the region immediately upstream of the I. orientalis ADH9091 open reading frame, an I. orientalis PDCl promoter, the S. pombe LYS4_D123N gene (having the nucleotide sequence SEQ ID NO: 12), the I. orientalis TAL terminator, the I. orientalis URA3 promoter, and the first 530 bp of the I. orientalis URA3 open reading frame.

Integration fragment P4-2, having nucleotide sequence SEQ ID NO: 13, contains the following elements, 5′ to 3′: a DNA fragment corresponding to the last 568 bp of the I. orientalis URA3 open reading frame, the I. orientalis URA3 terminator, the I. orientalis URA3 promoter, the I. orientalis TKL terminator, and a DNA fragment with homology for integration corresponding to the region immediately downstream of the I. orientalis ADH9091 open reading frame.

Strain P-3 is transformed simultaneously with integration fragments P4-1 and P4-2, using lithium acetate methods, to insert the S. pombe LYS4_D123Ngene at the ADH9091 locus. Integration occurs via three cross-over events: in the regions of the ADH9091 upstream homology, in the regions of the ADH9091 downstream homology and in the region of URA3 homology between SEQ ID NO: 11 and SEQ ID NO:13. Transformants are streaked to isolates and the correct integration of the cassette at the AHD9091 locus is confirmed in a strain by PCR. The PCR screening is performed using primers having nucleotide sequences SEQ ID NOs: 14 and 15 to confirm the 5′-crossover and SEQ ID NOs: 16 and 17 to confirm the 3′-crossover. That strain is grown and plated on FOA as before until the loopout of the URA3 marker from an isolate is confirmed by PCR.

That isolate is then transformed simultaneously with integration fragments P4-3 and P4-4 using LiOAc transformation methods, to insert a second copy of the S. pombe LYS4_D123N gene at the ADH9091 locus.

Integration fragment P4-3, having the nucleotide sequence SEQ ID NO: 18, contains the following elements, 5′ to 3′: a DNA fragment with homology for integration corresponding to the region immediately downstream of the I. orientalis ADH9091 open reading frame, an I. orientalis PDCl promoter, the S. pombe LYS4_D123N gene as found in SEQ ID NO: 12, the I. orientalis TAL terminator, the I. orientalis URA3 promoter, and the first 530 bp of the I. orientalis URA3 open reading frame.

Integration fragment P4-4, having the nucleotide sequence SEQ ID NO: 19, contains the following elements, 5′ to 3′: a DNA fragment corresponding to the last 568 bp of the I. orientalis URA3 open reading frame, the I. orientalis URA3 terminator, the I. orientalis URA3 promoter, the I. orientalis TKL terminator, and a DNA fragment with homology for integration corresponding to the region immediately upstream of the I. orientalis ADH9091 open reading frame.

Integration again occurs via three crossover events. Transformants are streaked to isolates and screened by PCR to identify a strain containing two copies of the S. pombe LYS4_D123N gene at the ADH9091 locus. The PCR screening to confirm the first copy is performed using primers having nucleotide sequences SEQ ID NOs: 14 and 15 to confirm the 5′-crossover and SEQ ID NOs: 16 and 17 to confirm the 3′-crossover. The PCR screening to confirm the second copy is performed using primers having nucleotide sequences SEQ ID NOs: 14 and 16 to confirm the 5′-crossover and SEQ ID NOs: 15 and 17 to confirm the 3′-crossover. That strain is grown and replated on FOA until a strain in which the URA3 marker has looped out is identified. That strain is designated strain P-4. The endogenous GPDI is attenuated with integration fragment 5 (having nucleotide sequence SEQ ID NO: 20) using lithium acetate methods as described before. This integration fragment contains the following elements, 5′ to 3′: a DNA fragment with homology for integration corresponding to the region immediately upstream of the I. orientalis GPD1 open reading frame, a PDC transcriptional terminator, the URA3 promoter, the I. orientalis URA3 gene, an additional URA3 promoter direct repeat for marker recycling and a DNA fragment with homology for integration corresponding to the region directly downstream of the I. orientalis GPD1 open reading frame. Successful transformants are selected on selection plates lacking uracil, confirmed by PCR using primers having nucleotide sequences SEQ ID NOs: 21 and 22 to confirm the 5′-crossover and SEQ ID NOs: 23 and 24 to confirm the 3′-crossover, and grown and plated on FOA as before until a strain in which the URA3 marker has looped out is identified. This strain is then transformed with an integration fragment having nucleotide sequence SEQ ID NO: 25. This integration fragment contains the following elements, 5′ to 3′: a DNA fragment with homology for integration corresponding to the region immediately upstream of the I. orientalis GPD1 open reading frame, the URA3 promoter, the I. orientalis URA3 gene, an additional URA3 promoter direct repeat for marker recycling a PDC transcriptional terminator, and a DNA fragment with homology for integration corresponding to the region directly downstream of the I. orientalis GPD1 open reading frame. Successful transformants are again selected on selection plates lacking uracil, and integration of the second GPD1 deletion construct confirmed by PCR using primers having nucleotide sequences SEQ ID NOs: 22 and 24 to confirm the 5′-crossover and SEQ ID NOs: 21 and 23 to confirm the 3′-crossover. Retention of the first GPD1 deletion construct is also reconfirmed by repeating the PCR reactions used to verify proper integration of integration fragment 5 above. Confirmed isolates are grown and plated until a strain in which the URA3 marker has looped out is identified as before. One such transformant which has a deletion of both native GPD genes, is designated Example 5-1.

In a similar way as the genetic modification methods described here, other the I. orientalis genes can be modified, deleted, or inserted into the genome in various combinations. These genes may include the I. orientalis genes that are homologous to the S. cerevisiae for ACO1 (homocitrate dehydrates), ACO2 (homocitrate dehydrates), LYS4 (homoaconitase), LYS12 (homoisocitrate dehydrogenase), LYS2 (alpha aminoadipate reductase), LYS9 (saccharopine dehydrogenase), LYS1 (saccharopine dehydrogenase, L-lysine forming), or the transcriptional regulatory genes as LYS14 and LYS80.

The Yeast AAA Lysine Biosynthesis Pathway is shown below (from http://pathway.yeastgenome.org/YEAST/NEW-IMAGE?type=PATHWAY&object=LYSINE-AMINOAD-PWY&detail-level=3&detail-level=2) gene shown are the Saccharomyces cerevisiae genes (NOTE The homoaconitase dehydration step has been modified to incorporate new findings from (Fazius F, Shelest E, Gebhardt P, Brock M. The fungal α-aminoadipate pathway for lysine biosynthesis requires two enzymes of the aconitase family for the isomerization of homocitrate to homoisocitrate. Mol Microbiol. 2012 December; 86(6):1508-30. doi: 10.1111/mmi.12076. Epub 2012 Nov. 6. PubMed PMID: 23106124; PubMed Central PMCID: PMC3556520)). The report showed that the homoaconitate dehydratase step is performed by ACO1 or ACO2 (preferred).

Saccharomyces cerevisiae: AAA Lysine Biosynthesis Pathway

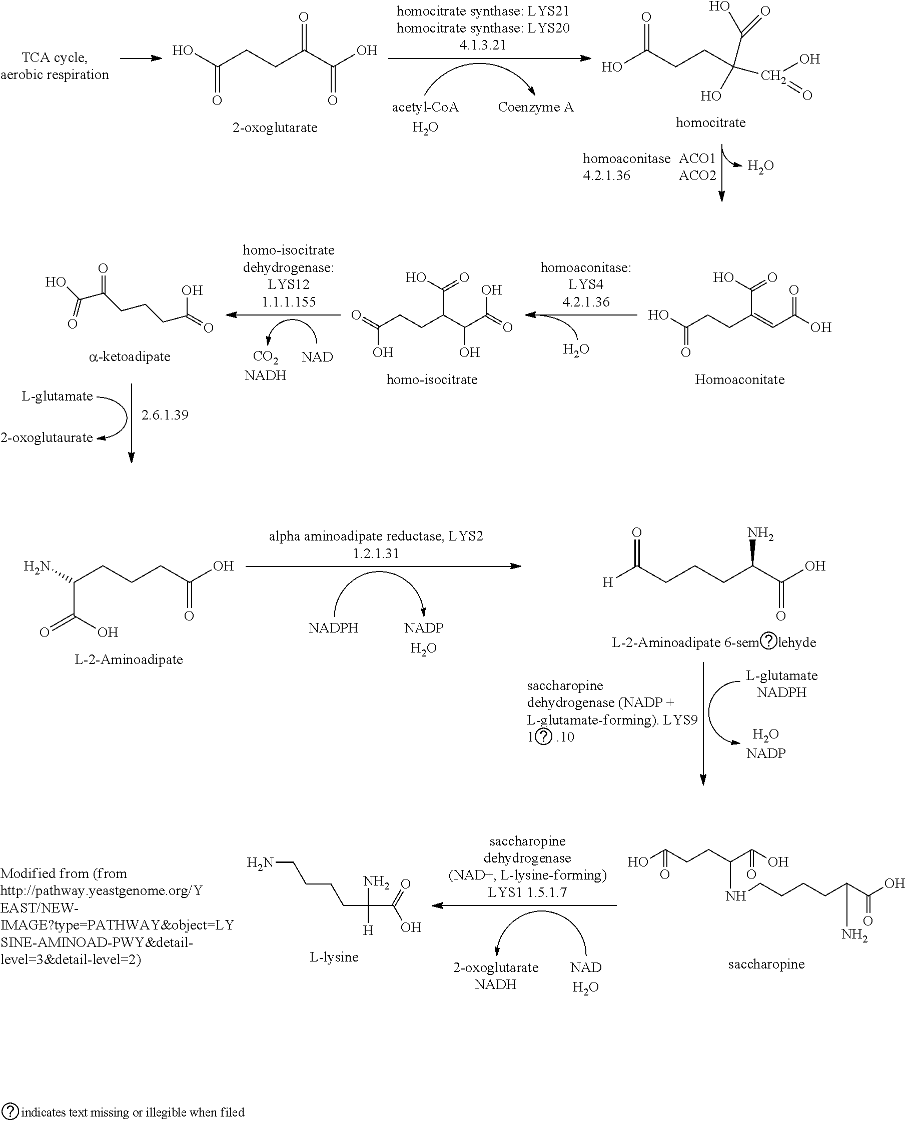

Also note: LYS20 and LYS21 have been shown to be important to regulation of this pathway as these enzymes often show feedback inhibition by lysine. In some embodiments, lysine insensitive variants of these genes would be used. For example, Feller et al. (Feller A, Ramos F, Piérard A, Dubois E. In Saccharomyces cerevisae, feedback inhibition of homocitrate synthase isoenzymes by lysine modulates the activation of LYS gene expression by Lys14p. Eur J Biochem. 1999 April; 261(1):163-70. PubMed PMID: 10103047.) describes mutations in LYS20 and LYS21 from strains that were isolated as being resistant to aminoethylcysteine, a toxic lysine analog. In addition this report also describes the transcriptional regulation of the lysine pathway via genes such as LYS14P, and ways of increasing alpha-ketoglutarate in Saccharomyces cerevisae via mutations in the LYS80 gene. Additionally, homocitrate synthase genes from other yeast could be used (Gasent-Ramirez J M, Benitez T. Lysine-overproducing mutants of Saccharomyces cerevisiae baker's yeast isolated in continuous culture. Appl Environ Microbiol. 1997 December; 63(12):4800-6. PubMed PMID: 9406398; PubMed Central PMCID: PMC168803.). For example, Bulfer et al. (Bulfer S L, Scott E M, Pillus L, Trievel R C. Structural basis for L-lysine feedback inhibition of homocitrate synthase. J Biol Chem. 2010 Apr. 2; 285(14):10446-53. doi: 10.1074/jbc.M109.094383. Epub 2010 Jan. 19. PubMed PMID: 20089861; PubMed Central PMCID: PMC2856251) describes several individual point mutations (D123N, E22Q, R288K, and Q364R) in a Schizosaccharomyces pombe LYS4 (a homocitrate synthase) that lead to less inhibition by lysine.

### Example 13—Synthetic Procedures and Catalytic Performance of Pt and Cu—Pd Nanocatalysts

### 1. Pt Nanoparticles

The platinum (Pt) nanoparticles were synthesized using the polyol method. More specifically, 0.1227 g of platinum chloride (PtC14, Sigma Aldrich, 99.9%) was diluted in anhydrous ethylene glycol (EG) (Sigma Aldrich, 99.8%). Subsequently, a solution of sodium hydroxide (NaOH, Sigma Aldrich, 97%) in ethylene glycol was added to adjust the pH of the solution to 11 while the final volume was 50 ml. The reactant mixture was vigorously stirred and heated under reflux at 160° C. for 3 hours. The resulting dark brown colloidal solution of Pt nanoparticles was cooled down to room temperature.

### 2. Cu—Pd Bimetallic Nanoparticles

The Cu—Pd bimetallic nanoparticles with the nominal atomic ratio of Cu:Pd=95:5 (Cu₉₅Pd₅) and 90:10 (Cu₉₀Pd₁₀) were prepared using a step synthesis procedure based on the polyol method. Briefly, first, a colloidal solution of copper nanoparticles was prepared. Second, the appropriate amount of the as-prepared Cu colloidal solution was mixed with a solution of palladium precursor salt in ethylene glycol. Third, the mixture of Cu colloids and Pd salt in ethylene glycol was refluxed, resulting in formation of bimetallic CuPd nanoparticles.

The detailed procedure is as follows:

1) 0.0984 g of copper nitrate (Cu(NO₃)₂, Alfa Aesar, 99%) was diluted in 30 ml of EG and the pH of the solution was adjusted to 11.1 using 30 ml of sodium hydroxide solution in (EG) (0.2 M). The resulting solution was refluxed for three hours at 190° C. under vigorous stirring and then cooled down to room temperature. The as-prepared colloidal solution of copper nanoparticles was used as the copper source for the synthesis of the bimetallic Cu—Pd Nanoparticles.
2) Appropriate amounts of palladium acetate (Pd(CH₃COO)₂, Sigma Aldrich, 99.98%) were diluted in ethylene glycol and 8 ml of the Cu colloidal solution were added. The pH of the mixture was adjusted to 11.2 using a NaOH/EG solution (0.2 M).
3) The mixture was stirred at room temperature for 1 hour and then refluxed at 196° C. for two hours. The resulting dark brown colloidal solution of Cu—Pd was then cooled to room temperature.

### 3. Preparation of Carbon Supported Nanocatalysts

The preparation of carbon supported nanocatalysts was carried out by mixing appropriate aliquots of the colloidal solution with carbon black (Vulcan-XC-72, CABOT Corp.) to obtain the supported catalysts with 1 and 3 weight (wt.) % metal loading in the case of the Pt supported on carbon (Pt/C) catalysts and 10 wt. % for Cu₉₅Pd₅/C and Cu₉₀Pd₁₀/C. The mixture of the nanoparticle solution and the carbon powder remained under vigorous stirring for three days and then was separated by centrifugation (10,000 rpm) and washed with deionized water. The centrifugation/washing cycle was repeated ten times to remove traces of ethylene glycol and NaOH. Finally, the obtained catalyst powders were dried in a freeze-dryer overnight.

### 4. Activation of Nanocatalysts

Prior to the catalytic tests the nanocatalysts were subject to an activation step: the desired amount of catalysts was transferred to a high-pressure (HP) reactor (Symyx Discovery Tools) and the following steps were performed:
a. Annealing at 180° C. under 400 psi of N₂ for 3 h.
b. Annealing at 180° C. under 200 psi of H₂ for 3 h.

### 5. Catalytic Tests

The activated nanocatalysts were tested for the catalytic conversion of lactone to adipic acid and other useful chemicals. The tested catalysts included:
1) 1 wt. % Pt/C
2) 3 wt. % Pt/C
3) 10 wt. % Pd₉₅Cu₅/C
4) 10 wt. % Pd₉₀Cu₁₀/C

The reaction was carried out at 180° C. with 1 mol % metal concentration for 16 hours under 450 psi of H₂.

### Example 14—Catalyzed Thermolysis of Various Starting Feed with No Added Hydrogen

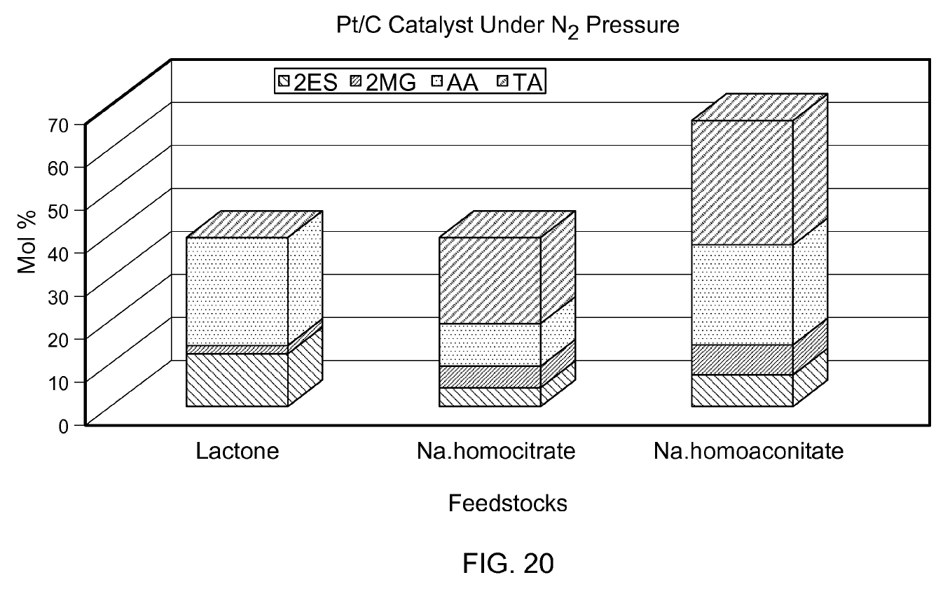

As shown in FIG. 20, combining homoaconitic acid with supported metal catalysts (1 mol % metal) and water at 180° C. for 16 hours under 450 psi N₂ resulted in significant production of decarboxylation products. FIG. 20 is the representative example when homocitric acid, homocitric acid lactone, or homoaconitic acid was used as the starting feed. Pt/C showed remarkable selectivity to adipic acid in the absence of added H₂ and in the presence of 450 psi of N₂ pressure. Sodium homocitrate and homoaconitate showed similar behaviour with Pt/C catalyst under the same conditions. Increased activity with sodium homoaconitate suggests stability with the pre-formed intermediate.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for making adipic acid, or a salt or ester thereof, the method comprising contacting homocitric acid, or a salt, ester, or lactone thereof, with a metal catalyst.

2. A method for making a compound of Formula I: 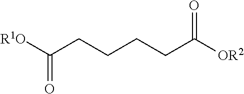
or a salt thereof,
wherein:
each R¹ and R² is individually selected from H and a protecting group;
the method comprising contacting a metal catalyst with a composition comprising a compound of Formula II: 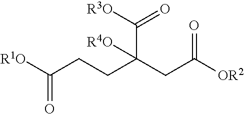
or a salt thereof,
wherein:
each R¹, R², R³, and R⁴ is individually selected from H and a protecting group.

3. A method for making a compound of Formula I: 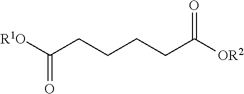
or a salt thereof,
wherein:
each R¹ and R² is individually selected from H and a protecting group;
the method comprising contacting a metal catalyst with composition comprising a compound of Formula III: 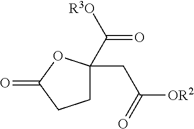
or a salt thereof,
wherein:
each R² and R³ is individually selected from H and a protecting group.

4. A method for making a compound of Formula I: 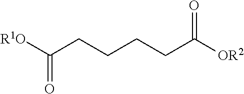
or a salt thereof,
wherein:
each R¹ and R² is individually selected from H and a protecting group;
the method comprising:
a) hydrogenolysis of a compound of Formula II: 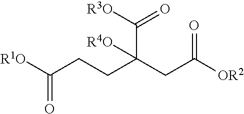
or a salt thereof,
wherein:
each R¹, R², R³, and R⁴ is individually selected from H and a protecting group;
to prepare a compound of Formula IV: 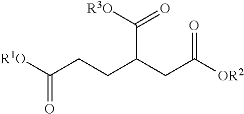
or a salt thereof,
wherein:
each R¹, R², R³, and R⁴ is individually selected from H and a protecting group; and
b) selective decarboxylation of the compound of Formula IV to make a compound of Formula I, or a salt thereof.

5. A method for making a compound of Formula I: 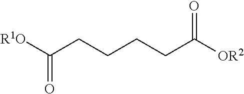
or a salt thereof,
wherein:
each R¹ and R² is individually selected from H and a protecting group;
the method comprising:
a) hydrogenolysis of a compound of Formula III: 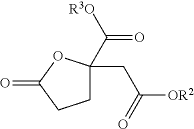
or a salt thereof,
wherein:
each R² and R³ is individually selected from H and a protecting group;
to prepare a compound of Formula IV: 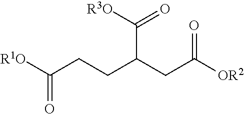
or a salt thereof,
wherein:
each R¹, R², R³, and R⁴ is individually selected from H and a protecting group; and
b) selective decarboxylation of the compound of Formula IV to make a compound of Formula I, or a salt thereof.

6. A method for making 2-ethylsuccinic acid, or a salt or ester thereof, the method comprising contacting homocitric acid, or a salt, ester, or lactone thereof, with a metal catalyst.

7. A method for making a compound of Formula V: 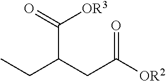
or a salt thereof,
wherein:
each R² and R³ is individually selected from H and a protecting group;
the method comprising contacting a metal catalyst with a composition comprising a compound of Formula II: 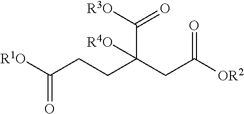
or a salt thereof,
wherein:
each R¹, R², R³, and R⁴ is individually selected from H and a protecting group.

8. A method for making a compound of Formula V: 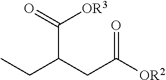
or a salt thereof,
wherein:
each R² and R³ is individually selected from H and a protecting group;
the method comprising contacting a metal catalyst with a composition comprising a compound of Formula III: 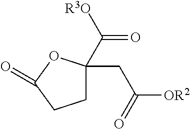
or a salt thereof,
wherein:
each R² and R³ is individually selected from H and a protecting group.

9. A method for making a compound of Formula V: 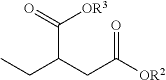
or a salt thereof,
wherein:
each R² and R³ is individually selected from H and a protecting group;
the method comprising:
a) hydrogenolysis of a compound of Formula II: 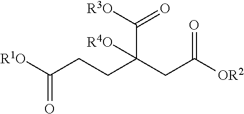
or a salt thereof,
wherein:
each R¹, R², R³, and R⁴ is individually selected from H and a protecting group;
to prepare a compound of Formula IV: 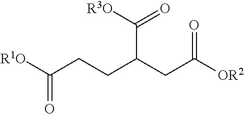
or a salt thereof,
wherein:
each R¹, R², R³, and R⁴ is individually selected from H and a protecting group; and
b) selective decarboxylation of the compound of Formula IV to make a compound of Formula V, or a salt thereof.

10. A method for making a compound of Formula V: 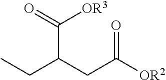
or a salt thereof,
wherein:
each R² and R³ is individually selected from H and a protecting group;
the method comprising:
a) hydrogenolysis of a compound of Formula III: 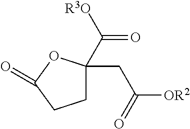
or a salt thereof,
wherein:
each R² and R³ is individually selected from H and a protecting group;
to prepare a compound of Formula IV: 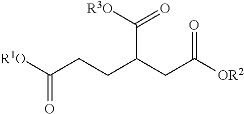
or a salt thereof,
wherein:
each R¹, R², R³, and R⁴ is individually selected from H and a protecting group; and
b) selective decarboxylation of the compound of Formula IV to make a compound of Formula V, or a salt thereof.

11. A method for making 2-methylpentanedioic acid, or a salt or ester thereof, the method comprising contacting homocitric acid, or a salt, ester, or lactone thereof, with a metal catalyst.

12. A method for making a compound of Formula VI: 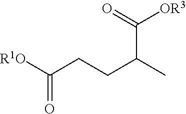
or a salt thereof,
wherein:
each R¹ and R³ is individually selected from H and a protecting group;
the method comprising contacting a metal catalyst with a composition comprising a compound of Formula II: 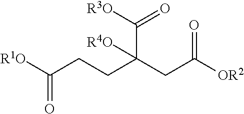
or a salt thereof,
wherein:
each R¹, R², R³, and R⁴ is individually selected from H and a protecting group.

13. A method for making a compound of Formula VI: 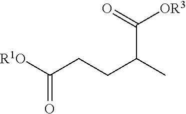
or a salt thereof,
wherein:
each R¹ and R³ is individually selected from H and a protecting group;
the method comprising contacting a metal catalyst with a composition comprising a compound of Formula III: 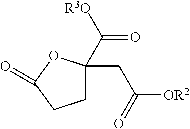
or a salt thereof,
wherein:
each R² and R³ is individually selected from H and a protecting group.

14. A method for making a compound of Formula VI: 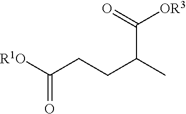
or a salt thereof,
wherein:
each R¹ and R³ is individually selected from H and a protecting group;
the method comprising:
a) hydrogenolysis of a compound of Formula II: 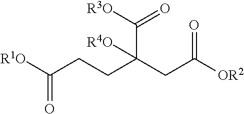
or a salt thereof,
wherein:
each R¹, R², R³, and R⁴ is individually selected from H and a protecting group;
to prepare a compound of Formula IV: 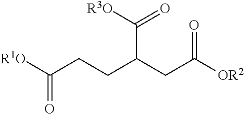
or a salt thereof,
wherein:
each R¹, R², R³, and R⁴ is individually selected from H and a protecting group; and
b) selective decarboxylation of the compound of Formula IV to make a compound of Formula VI, or a salt thereof.

15. A method for making a compound of Formula VI: 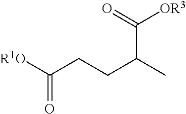
or a salt thereof,
wherein:
each R¹ and R³ is individually selected from H and a protecting group;
the method comprising:
a) hydrogenolysis of a compound of Formula III: 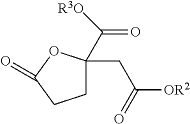
or a salt thereof,
wherein:
each R² and R³ is individually selected from H and a protecting group;
to prepare a compound of Formula IV: 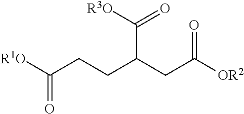
or a salt thereof,
wherein:
each R¹, R², R³, and R⁴ is individually selected from H and a protecting group; and
b) selective decarboxylation of the compound of Formula IV to make a compound of Formula VI, or a salt thereof.

16. A method for making a composition comprising two or more compounds selected from the group consisting of: adipic acid, 1,2,4-butanetricarboxylic acid, 2-ethylsuccinic acid, and 2-methylpentanedioic acid, or a salt or ester thereof, the method comprising contacting homocitric acid, or a salt, ester, or lactone thereof, with a metal catalyst.

17. A method for making a composition comprising two or more compounds selected from the group consisting of: 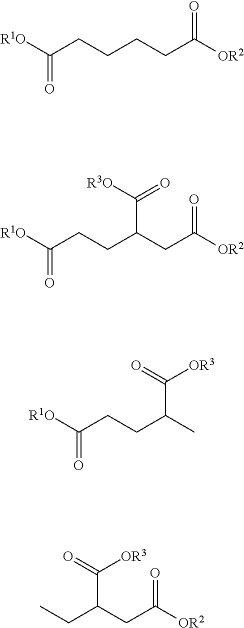
or a salt thereof,
wherein:
each R¹, R², and R³ is individually selected from H and a protecting group;
the method comprising contacting a metal catalyst with a composition comprising a compound of Formula II: 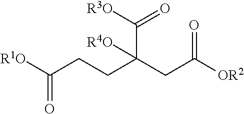
or a salt thereof,
wherein:
each R¹, R², R³, and R⁴ is individually selected from H and a protecting group.

18. A method for making a composition comprising two or more compounds selected from the group consisting of: 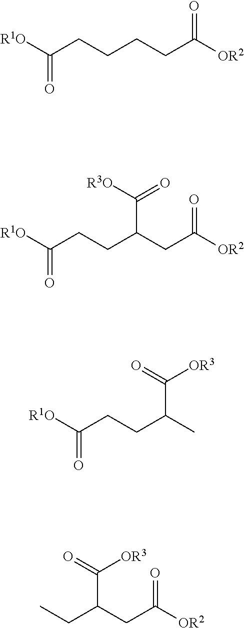
or a salt thereof,
wherein:
each R¹, R², and R³ is individually selected from H and a protecting group;
the method comprising contacting a metal catalyst with a composition comprising a compound of Formula III: 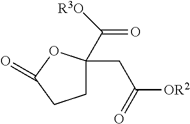
or a salt thereof,
wherein:
each R² and R³ is individually selected from H and a protecting group.

19. A method for making a composition comprising two or more compounds selected from the group consisting of: 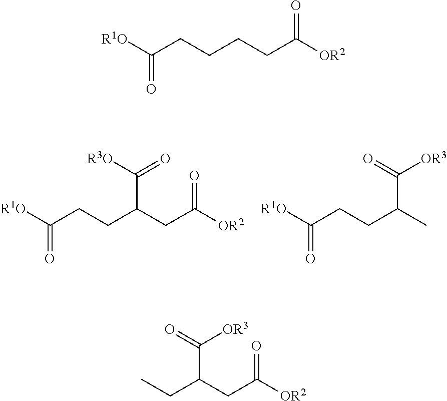
or a salt thereof,
wherein:
each R¹, R², and R³ is individually selected from H and a protecting group;
the method comprising:
a) hydrogenolysis of a compound of Formula II: 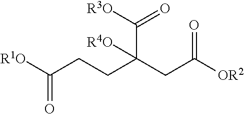
or a salt thereof,
wherein:
each R¹, R², R³, and R⁴ is individually selected from H and a protecting group;
to prepare a compound of Formula IV: 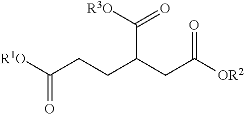
or a salt thereof,
wherein:
each R¹, R², R³, and R⁴ is individually selected from H and a protecting group; and
b) selective decarboxylation of the compound of Formula IV to prepare the composition.

20. A method for making two or more compounds selected from the group consisting of: 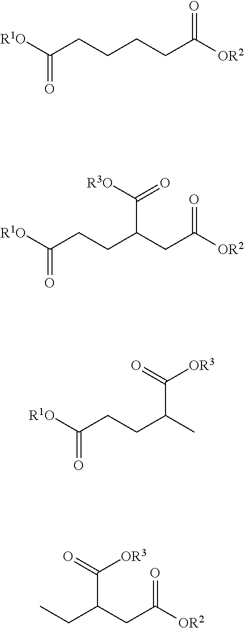
or a salt thereof,
wherein:
each R¹, R², and R³ is individually selected from H and a protecting group;
the method comprising:
a) hydrogenolysis of a compound of Formula III: 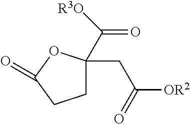
or a salt thereof,
wherein:
each R² and R³ is individually selected from H and a protecting group;
to prepare a compound of Formula IV: 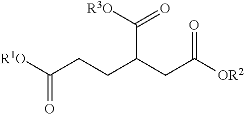
or a salt thereof,
wherein:
each R¹, R², R³, and R⁴ is individually selected from H and a protecting group; and
b) selective decarboxylation of the compound of Formula IV to prepare the composition.

21. The method of any one of claims 1-20, wherein the metal catalyst is a heterogeneous catalyst.

22. The method of any one of claims 1-21, wherein the metal catalyst comprises a metal selected from the group consisting of Ni, Pd, Pt, Re, Ag, Au, Cu, Zn, Rh, Ru, Bi, Fe, Co, Os, Ir, V, and mixtures of two or more thereof.

23. The method of any one of claims 1-22, wherein the metal catalyst comprises a metal selected from the group consisting of Pd and Pt.

24. The method of claim 23, wherein the metal catalyst comprises Pd.

25. The method of any one of claims 1-22, wherein the metal catalyst is a bimetallic catalyst.

26. The method of claim 25, wherein the metal catalyst comprises Pd and Cu.

27. The method of any one of claims 1-26, wherein the metal catalyst is a nanocatalyst.

28. The method of any one of claims 1-27, wherein the metal catalyst is a supported catalyst.

29. The method of any one of claims 1-28, wherein the metal catalyst comprises a promoter.

30. The method of method 29, wherein the promoter comprises sulfur.

31. The method of any one of claims 1-30, wherein the method is performed at a temperature of at least about 100° C.

32. The method of any one of claims 1-31, wherein the method is performed at a temperature of about 100° C. to about 200° C.

33. The method of any one of claims 1-32, wherein the method is performed at a temperature of about 150° C. to about 180° C.

34. The method of any one of claims 1-33, wherein the metal catalyst is activated prior to the contacting.

35. The method of claim 34, wherein the metal catalyst is activated under hydrogen gas.

36. The method of any one of claim 34 or 35, wherein the metal catalyst is activated at a temperature of about 100° C. to about 200° C.

37. A method for making adipic acid, or a salt or ester thereof, the method comprising contacting homocitric acid lactone with a Pd(S)/C catalyst.

38. A method for making a compound of Formula I: 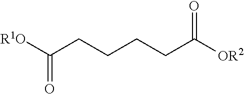
or a salt thereof,
wherein:
each R¹ and R² is individually selected from H and a protecting group;
the method comprising contacting a Pd(S)/C catalyst with composition comprising a compound of Formula III: 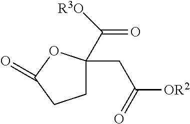
or a salt thereof,
wherein:
each R² and R³ is individually selected from H and a protecting group.

39. A composition comprising two or more compounds selected from the group consisting of: adipic acid, 1,2,4-butanetricarboxylic acid, 2-ethylsuccinic acid, and 2-methylpentanedioic acid, or a salt or ester thereof.

40. A composition comprising two or more compounds selected from the group consisting of: 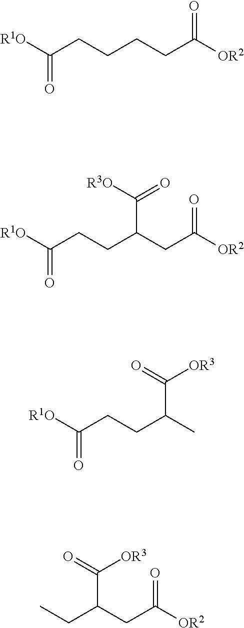
or a salt thereof,
wherein:
each R¹, R², and R³ is individually selected from H and a protecting group.

41. A method for making the composition according to claim 40 comprising;
culturing an recombinant acidophilic yeast in a fermentation broth, wherein the fermentation broth comprises homocitrate lactone;
contacting the homocitrate lactone with a metal catalyst; and
producing the composition according to claim 40.

42. A method of making adipic acid or a salt thereof, comprising:
contacting a genetically engineered microorganism that overproduces a product selected from homocitrate, homoaconitate or combinations thereof with a carbohydrate source;
separating the homocitrate, homoaconitate or combinations thereof; and
catalytically converting the homocitrate, homoaconitate or combinations thereof to adipic acid.


SEQUENCE LISTING (25 sequences)

SEQ ID NO: 1 - DNA, 4910 bp, Artificial Sequence
  Description of Artificial Sequence Synthetic URA 3 gene disruption fragment
ctcaaaactatttaattagttaattgtataaactgtatgtcattataaacagggaaggttgacattgtctagcggcaatcattgtctcatttggttcattaactttggttctgttcttggaaacgggtaccaactctctcagagtgcttcaaaaatttttcagcacatttggttagacatgaactttctctgctggttaaggattcagaggtgaagtcttgaacacaatcgttgaaacatctgtccacaagagatgtgtatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcaccctgcagggtacgtagcatgcactcgcaagctgtgccatcgcccaacggttaattataagaaatcaacatcagccaacaactattttcgtccccctcttttcagtggtaacgagcaattacattagtaagagactattttcttcagtgatttgtaattttttttcagtgatttgtaattctttctcgaaatatgcgggcttaacttatccggacattcactacatgcaaggaaaaacgagaaccgcggagatttcctcagtaagtaacaatgatgatctttttacgcttcatcatcactttccaaagttctaagctataagttcaagcctagatacgctgaaaaactcctgaccaacaatgtaaagaaaacaattacaattgtaaggttgaaaacatctaaaaatgaaatattttattgtacatgcacaccctgatagtcattctcttacttcatccctgaaagacgtggctgtacaagagttggaatcgcaaggtcatgaggttaaagttagtgatctttatgctcaaaagtggaaggcctcaatagaccgtgacgacttcgagcagcttttcgcaagaagagaggttaaaaataccccaagcttcttatgaagcgtatgccagaggagcattaacaaaagacgtaaatcaggaacaggaaaaacttatttgggcggactttgtcattttgtcgtttcctatatggtggtcttctatgccggctagtcgaccccctcgaccccctcgagcgatctcgagatttgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgnaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacaatgtttgctttctactttctcaccgcatgcaccactttgaagggtgttttcggagtttctccgagttacaatggtcttggtctcaccccacagatgggttgggacagctggaatacgtttgcctgcgatgtcagtgaacagctacttctagacactgctgatagaatttctgacttggggctaaaggatatgggttacaagtatgtcatcctagatgactgttggtctagcggcagggattccgacggtttcctcgttgcagacaagcacaaatttcccaacggtatgggccatgttgcagaccacctgcataataacagctttcttttcggtatgtattcgtctgctggtgagtacacctgtgctgggtaccctgggtctctggggcgtgaggaagaagatgctcaattctttgcaaataaccgcgttgactacttgaagtatgataattgttacaataaaggtcaatttggtacaccagacgtttcttaccaccgttacaaggccatgtcagatgctttgaataaaactggtaggcctattttctattctctatgtaactggggtcaggatttgacattttactggggctctggtatcgccaattcttggagaatgagcggagatattactgctgagttcacccgtccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaatttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttctataggttgagaccctcggcttaagctcaatgttgagcaaagcaggacgagaaaaaaaaaaataatgattgttaagaagttcatgaaaaaaaaaaggaaaaatactcaaatacttataacagagtgattaaataataaacggcagtataccctatcaggtattgagatagttttatttttgtaggtatataatctgaagcctttgaactattttctcgtatatatcatggagtatacattgcattagcaacattgcatactagtcactcgcaagctgtgccatcgcccaacggttaattataagaaatcaacatcagccaacaactattttcgtccccctcttttcagtggtaacgagcaattacattagtaagagactattttcttcagtgatttgtaattttttttcagtgatttgtaattctttctcgaaatatgcgggctwaamtaatccggacattcactacatgcaaggaaaaacgagaaccgcggagatttcctcagtaagtaacaatgatgatctttttacgcttcatcatcactttccaaagttctaagctataagttcaagcctagatacgctgaaaaactcctgaccaacaatgtaaagaaaacaattacaattgtaaggttgaaaacatctaaaaatgaaatattttattgtacatgcacaccctgatagtcattctcttacttcatccctgaaagacgtggctgtacaagagttggaatcgcaaggtcatgaggttaaagttagtgatctttatgctcaaaagtggaaggcctcaatagaccgtgacgacwwmaaaaaamaaamrmaagaagagaggttaaaaataccccaagcttcttatgaagcgtatgccagaggagcattaacaaaagacgtaaatcaggaacaggaaaaacttatttgggcggactttgtcattttgtcgtttcctatatggtggtcttctatgccggctagcggccgggcaacaaagcctcccagatttgatanattttcaatttgtgctttgaatcatgacttccacctgtttggtccgcaagaacacgtaaatgcgcaatttgtttctcccttctgcttaaaaaccatgcacctttaatattatctggaaagataaagaacagaattgttgcgtagaaacaagtagcagagccgtaaatgagaaaaatatacttccaagctggtaatttcccctttattagtccaatacagtgtccgaagaccccaccaagaataccagcaagggtgttgaaatataatgtagatcttagtggttgttctgatttcttccaccacattccgctaataatcataaaagacggtaatattccggcttcaaatacgccaagaaaaaacctcacggtaaccaaaccaccaaagctatgacatgcagccatgcacataagtaagccgccccaaatgaacaaacaaatagacacaaatttgccaattctaactcgtggcaacaaaaaaaaggatatgaactcacctaataaataaccgaaataaaaagtagaagcaactgtggaaaattgagaaccatgtaaatttgtgtcttctttcaatgtataaacagccgcaatacctaggg

SEQ ID NO: 2 - DNA, 22 bp, Artificial Sequence
  Description of Artificial Sequence Synthetic primer
gcaactgatgttcacgaatgcg

SEQ ID NO: 3 - DNA, 20 bp, Artificial Sequence
  Description of Artificial Sequence Synthetic primer
ttgccgttgcagcaaatctc

SEQ ID NO: 4 - DNA, 21 bp, Artificial Sequence
  Description of Artificial Sequence Synthetic primer
acggcagtataccctatcagg

SEQ ID NO: 5 - DNA, 20 bp, Artificial Sequence
  Description of Artificial Sequence Synthetic primer
aatgatccatggtccgagag

SEQ ID NO: 6 - DNA, 3704 bp, Artificial Sequence
  Description of Artificial Sequence Synthetic PDC gene disruption fragment
cccccagttgttgttgcaattaacaaatttgctaccgacaccgagaaggaaattgagaccattagagaagaagccatcaaggctggtgcatttgatgctgttgagtcagaccattggtcacaaggtggtaagggtgcaatcaagttagctgaggcaattgtacgtgctaccgaggaaagaccgttggaagaaagtcaacctcctaactatctttattcattagatggttcgttagaagatagactaagaacaattgccaccaagatgtatggagcaaaagatattgaactatctgagttggccaagaaacagattgaagagtatgagagtcaaggttttggcaagctgcctgtttgtattgcaaagacgcaatattctctctcccatgatccaacattgaaaggtgttccaaaggatttcatcttcccaatcagagaagttagaataagtgcaggagcaggatatttatatgcactagctgcaaagatcatgacaattccaggtctatcaacttatgccggatttatgaatgttgaagtcaacgaagatggtgagattgatggattgttttagtttttattataaaattatatattattcttaattacatatcacccttctatcagggaagggagaaacgaaaatagagagtgacctatccaagctcgggggtctaagttttaatggcccagggaatcattacttttttttctcaatccttgatggataaaagtattacatacgtacaggattgtgtattagtgtatttcgttatatgattaaacaaagtttatagattgtaaagtagacgtaaagtttagtaattcattttaatgttcattttacattcagatggcggccgcggatccagatcccccggggcgttgaagatctattctccagcaattaaatttgtgaagaataactggtatagagtacttcctttaaaaacatgtccgtgcaccaagaaaaaaaaaaagtttgaaaaattgtatgtcgacgaatttcagcattttcatttcaaggcgatattatgtttcactaaactcaggacaggaatatactaagaataactacaacatacacacaacataagccaagatggatcaacttaactaccaagaacaacaacaatttcaaaagatcgttgaacaaaagcaaatggctgatttcatgaggctatctgcagatacgcggaacaatcaatcgataatgatttgactgataaagaaaaccatacttttgtttatgtttattagttatcgctttgctacattaaaaattcacatactaaagcctttgttaaacaactttttctaaatcttaagattttactctatctagtttttttggttgtaggtgaacgtaaagtacctcatttatttttttttttttgcttgtgtaattcttttcatgcttatttaaactagtgtacatgtatcaaatctttgtgtaagaatcatttaaatctgtttaaataagcattccaaccagcttgttggtatcttttagcttgctctataggatctcttccttgaccgtacaaacctctaccaacaattatgatatccgttccagtctttacaacttcatcaacagttctatattgttgaccaagtgcatcacctttgtcatctaaaccaacccctggagtcataatgatccagtcaaaaccttcttctctaccgcccatatcgtgttgcgcaataaaaccaatgacaaactctttatcagatttagcaatttctactgttttttctgtatattcaccatatgctaaagaaccctttgatgataactcagcaagcattagcaaacctctaggttcactggttgtttcttgggctgcctccttcaagccagaaacaatacctgcacccgttacaccatgtgcattagtgatgtcagcccattcggcaatacggaagacaccagatttatattgatttttaacagtgttaccaatatcagcaaattttctatcttcaaaaatcataaaattatgtttcttggcaagctccttcaaaggcaacacagttccttcatacgtaaaatcagaaacaatatcgatgtgtgttttaactagacagatgtaaggaccaatagtgtccaaaatagagagaagcttttcagtttcagtaatatccaatgatgcacaaaggttagacttcttttcctccatgatggagaaaagtctcctagcaacaggggaagtgtgtgattctgatctttctttgtatgacgccatccttgacaaacaaactactttattaaagcgttgaagatctattctccagcaattaaatttgtgaagaataactggtatagagtacttcctttaaaaacatgtccgtgcaccaagaaaaaaaaaaagtttgaaaaattgtatgtcgacgaatttcagcattttcatttcaaggcgatattatgtttcactaaactcaggacaggaatatactaagaataactacaacatacacacaacataagccaagatggatcaacttaactaccaagaacaacaacaatttcaaaagatcgttgaacaaaagcaaatggctgatttcatgaggctatgaattcttttattataaaattatatattattcttaattacatatcacccttctatcagggaagggagaaacgaaaatagagagtgacctatccaagcttgggggtctaagttttaatggcccagggaatcattacttttttttctcaatccttgatggataaaagtattacatacgtacaggattgtgtattagtgtatttcgttatatgattaaacaaagtttatagattgtaaagtagacgtaaagtttagtaattcattttaatgttcattttacattcagatgttaattaaggcctcgagggatccgcggccgctatttttgtgttttgctgtgttttgttttattttgttttattgggaagaaaatatataataatagaatattatattaacaaataattaaagaagctcaactgttattagaataaatgggttctccgtgtcctttttatacgccttctccgaaaagaaaaaaaccatcgtatcatttgtagcccacgccacccggaaaaaccaccattgtcctcagcagtccgcaaaaatatggatgcgctcaatcaatttccctcccccgtcaatgccaaaaggataacgacacactattaagagcgcatcatttgtaaaagccgaggaagggggatacgctgaccgagacgtctcgcctcactctcggagctgagccgccctccttaagaaattcatgggaagaacacccttcgcggcttctgaacggctcgccctcgtccattggtcacctcacagtggcaactaataaggacattatagcaatagaaattaaaatggtgcacagaaatacaataggatcgaataggataggatacaataagatacggaatattagactatactgtgatacggtacgctacgatacgctacgatacgatacgatagaggataccacggatataacgtagtgttatttttcattattggggttttttttctgtttgaattttccacgtcaagagtatcccatctgacaggaaccgatggactcgtcacagtacctatcgcccgagttcaatccatggacgctgcgggtgaaggatcttcgcccgctgttggcaagccatgggatcagggcgtcgccaagggacgggcc

SEQ ID NO: 7 - DNA, 22 bp, Artificial Sequence
  Description of Artificial Sequence Synthetic primer
gaagagacgtacaagatccgcc

SEQ ID NO: 8 - DNA, 47 bp, Artificial Sequence
  Description of Artificial Sequence Synthetic primer
ggataaaagtattacatacgtacaggattgtgtattagtgtatttcg

SEQ ID NO: 9 - DNA, 22 bp, Artificial Sequence
  Description of Artificial Sequence Synthetic primer
taggaatggtgcatcatccaac

SEQ ID NO: 10 - DNA, 27 bp, Artificial Sequence
  Description of Artificial Sequence Synthetic primer
gctggagaatagatcttcaacgccccg

SEQ ID NO: 11 - DNA, 4106 bp, Artificial Sequence
  Description of Artificial Sequence Synthetic LYS4 D123N gene integration fragment
ctaagtagtggtgttggtgaactcaagatggactctttaggtaattatattcttgaatagttgtgtaaagcgaatatgcaaatagatttgttttataattatgcatctctttgaaagaggtttagaggcaaagttcttgcatacaatattgtgattgttttaatgtcattcttgattttcataaagagattaaaaaaaaaaaaaaaaaacttataaaattgagtagaaccatttatatataagacaaagattgtctgtattagtcctcaacacactaaaccttacatacttagggtaaatttgctaatagagtgatatgttcatgagaactccaacgacaacacaaccacctatttgcacaacaaacaccattgtcgcacgctgcgcgccctagaagtagaaagaaagggaaatgacattaagagaatcataccccgtgcccgtaacgccgaaaaaatcacaccccgtcccccacaccttaaaacctcaaccgcttaacaccgccacaccctttctctttataaacgccgtttgcattactcattcttcttataaaccgcaccccccaaaacgcggaatagcttcaaccccccaatcagatatgagtttcccgggaaacccgcttttcccgacagccccacaaggggttggtctataaaagaggacgttttccccgtcatcgagattgaagattcttacaggcccatttattcaaattggagttgattcttcttgtctttactttctttctctctttttcttccttttttaatattatcttttgtcaagcctggttccctaagttgaactctcttttcttgtgatcctcctatatagatacgccttgccaaatgcggccgcgagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaattctagataaaatgagtgtctctgaggcaaacggaaccgaaactatcaaacctccaatgaacggaaacccatacggacctaatccatctgacttcttgtccagagttaacaacttctcaatcatcgaaagtaccttgagagaaggagagcaattcgctaacgcctttttcgatactgagaagaaaattcagatcgcaaaggctcttgataacttcggagttgactacatcgaattgacttccccagttgcttcagaacaaagtagacaggactgtgaggctatctgcaaattgggtcttaagtgtaaaattttgactcatatcagatgccacatggatgacgctagagttgccgtcgaaactggtgttgatggagtcaacgttgtcattggtacatctcaatacttgagaaagtactctcatggtaaagatatgacctacatcatcgactctgctactgaggttattaattttgtcaagtccaaaggtatcgaagttagattttcttccgaggattctttcagatccgatcttgttgacttgctttctttgtacaaggctgttgataaaattggtgttaacagagtcggaatcgcagacaccgttggttgtgctactcctagacaagtctacgatcttatcagaacattgagaggagttgtctcctgtgatattgaatgccatttccacaacgacaccggtatggccattgcaaatgcttactgcgctttggaagccggagcaactcatatcgatacatcaatcttgggtatcggagagagaaatggtatcactccattgggagcattgcttgctagaatgtacgttacagatagagaatacatcacccacaagtacaaacttaaccaattgagagaacttgagaatttggttgctgacgccgttgaggtccagattccttttaacaattacatcactggaatgtgtgccttcacacataaggcaggtattcatgcaaaagctatccttgctaacccatcaacttacgaaattttgaagcctgaggatttcggaatgtcaagatatgttcatgtcggtagtagattgacaggatggaacgctattaagagtagagccgaacaattgaatcttcacttgacagacgcccaggcaaaagagttgaccgttagaattaagaaacttgccgatgtcagaactttggctatggatgacgttgacagagtcttgagagaataccacgcagacttgtccgacgcagatagaatcactaaggaagcctccgcctaattaattctgtctttgattttcttatgttattcaaaacatctgccccaaaatctaacgattatatatattcctacgtataactgtatagctaattattgatttatttgtacataaaaaccacataaatgtaaaagcaagaaaaaaaataactaaggagaaggatcaatatctcatttataatgctcgccaaagcagcgtacgtgaattttaatcaagacatcaacaaatcttgcaacttggttatatcgcttcttcacccactcacccgcttttctacattgttgaacacaaatatatacaggggtatgtctcaaggtcaagtgcagtttcaacagagactacctcaaggtacctcttcagaaatgcagaacttcactcttgatcagattttctccgaattaaaggtttaaacatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgttgag

SEQ ID NO: 12 - DNA, 1257 bp, Artificial Sequence
  Description of Artificial Sequence Synthetic codon optimized S Pombe LYS4 D123N
atgagtgtctctgaggcaaacggaaccgaaactatcaaacctccaatgaacggaaacccatacggacctaatccatctgacttcttgtccagagttaacaacttctcaatcatcgaaagtaccttgagagaaggagagcaattcgctaacgcctttttcgatactgagaagaaaattcagatcgcaaaggctcttgataacttcggagttgactacatcgaattgacttccccagttgcttcagaacaaagtagacaggactgtgaggctatctgcaaattgggtcttaagtgtaaaattttgactcatatcagatgccacatggatgacgctagagttgccgtcgaaactggtgttgatggagtcaacgttgtcattggtacatctcaatacttgagaaagtactctcatggtaaagatatgacctacatcatcgactctgctactgaggttattaattttgtcaagtccaaaggtatcgaagttagattttcttccgaggattctttcagatccgatcttgttgacttgctttctttgtacaaggctgttgataaaattggtgttaacagagtcggaatcgcagacaccgttggttgtgctactcctagacaagtctacgatcttatcagaacattgagaggagttgtctcctgtgatattgaatgccatttccacaacgacaccggtatggccattgcaaatgcttactgcgctttggaagccggagcaactcatatcgatacatcaatcttgggtatcggagagagaaatggtatcactccattgggagcattgcttgctagaatgtacgttacagatagagaatacatcacccacaagtacaaacttaaccaattgagagaacttgagaatttggttgctgacgccgttgaggtccagattccttttaacaattacatcactggaatgtgtgccttcacacataaggcaggtattcatgcaaaagctatccttgctaacccatcaacttacgaaattttgaagcctgaggatttcggaatgtcaagatatgttcatgtcggtagtagattgacaggatggaacgctattaagagtagagccgaacaattgaatcttcacttgacagacgcccaggcaaaagagttgaccgttagaattaagaaacttgccgatgtcagaactttggctatggatgacgttgacagagtcttgagagaataccacgcagacttgtccgacgcagatagaatcactaaggaagcctccgcctaa

SEQ ID NO: 13 - DNA, 2592 bp, Artificial Sequence
  Description of Artificial Sequence Synthetic C-term URA3 gene integration fragment
ctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgcgtttggcgcgccatctaatagtttaatcacagcttatagtctactatagttttcttttttaaacattgttgtattttgtcccccccctctaattgatgatgattatcctataagaatccaataaaacgatggaaactaataccctctcctttgtcatgtggtctttagtatttcttgaacattggctctgatttctcgactttatagtcctattaaaatcgctgttagttctcgatcgttgtatctcgtttcttgtctctttggtggatgattttgcgtgcgaacatgtttttttccctttctctcaccatcatcgtgtagttcttgtcaccatcccccccaccccttccttctctcattgattctataagagcttatccacagaggtgcagtaacgaggtagtttaaccttcgagtggatcaaaatgtcacacaggcctgcggccgctaccataatgtatgcgttgagcctcttgcaccttctttattaggaaatcagttgaaaaatttccggattgtctttattattggcccatttttttttggtcacacctttatttttgtacacttctcgggcaaagcaaaaactatagtaccggataggcctttataaaactccagtgtgtatgattttagttggtgtgccatctacacgttctcttagtttctttatcatgtcacagaaagcaagcatgcaaacccttacaaaaaataacaacatacaaatgcctaaacaactggactataatgatggtgagtcagttacgaaaagagcaagtgggttaatacgatttcgtaagggacagtctgaggaagactacaattttcaaaaggagcagttctggtccacgggtcctttagtacagaatcacacatttgtgactgaatttgttgaaaagtttattgaaaacacaattagtgaagattattcaatcacagatagatcgaaaatagaacgtgaaacaatcatacacggattggagaagctgtattttcaaagggaatatgagcgatgtctaaaagatgttcaactattgaaggacaatatcgataagttcaatcctaatttggatcttaatgaaaagaatttataatgagctgaattatatttcttggatgtgcatcaaaaagatccatgagagtaacgaaaagaaactgggggaaatctaataatttacaatttcaatatacacttctatatcctttaatgtaatggctttataaataaacacgaacttctacagcaccgacgtttctttttcttaccagctcctcttcttcttcttcttcttcttcttcttcttcttcttcttcttcttcttcttcttcttcttcttctttcttaccatcattgccattttccttttttcttatttgctcttgatcctctgttttttcaatttggacaaactcatctaatacaccaacacttttagggcccccgc

SEQ ID NO: 14 - DNA, 22 bp, Artificial Sequence
  Description of Artificial Sequence Synthetic primer
ggaggaatggaacagtgatgac

SEQ ID NO: 15 - DNA, 31 bp, Artificial Sequence
  Description of Artificial Sequence Synthetic primer
caagagtatcccatctgacaggaaccgatgg

SEQ ID NO: 16 - DNA, 20 bp, Artificial Sequence
  Description of Artificial Sequence Synthetic primer
cacagaggtgcagtaacgag

SEQ ID NO: 17 - DNA, 27 bp, Artificial Sequence
  Description of Artificial Sequence Synthetic primer
catcactgttaaaggaatgggtaaatc

SEQ ID NO: 18 - DNA, 4241 bp, Artificial Sequence
  Description of Artificial Sequence Synthetic LYS4 D123N gene inserter fragment
ctaaaagtgttggtgtattagatgagtttgtccaaattgaaaaaacagaggatcaagagcaaataagaaaaaaggaaaatggcaatgatggtaagaaagaagaagaagaagaagaagaagaagaagaagaagaagaagaagaagaagaagaagaagaagaagaggagctggtaagaaaaagaaacgtcggtgctgtagaagttcgtgtttatttataaagccattacattaaaggatatagaagtgtatattgaaattgtaaattattagatttcccccagtttcttttcgttactctcatggatctttttgatgcacatccaagaaatataattcagctcattataaattcttttcattaagatccaaattaggattgaacttatcgatattgtccttcaatagttgaacatcttttagacatcgctcatattccctttgaaaatacagcttctccaatccgtgtatgattgtttcacgttctattttcgatctatctgtgattgaataatcttcactaattgtgttttcaataaacttttcaacaaattcagtcacaaatgtgtgattctgtactaaaggacccgtggaccagaactgctccttttgaaaattgtagtcttcctcagactgtcccttacgaaatcgtattaacccacttgctcttttcgtaactgactcaccatcattatagtccagttgtttaggcatttgtatgttgttattttttgtaagggtttgcatgcttgctttctgtgacatgataaagaaactaagagaacgtgtagatggcacaccaactaaaatcatacacactggagttttataaaggcctatccggtactatagtttttgctttgcccgagaagtgtacaaaaataaaggtgtgaccaaaaaaaaatgggccaataataaagacaatccggaaatttttcaactgatttcctaataaagaaggtgcaagaggctcaacgcatacattatggtagcggccgcgagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaattctagataaaatgagtgtctctgaggcaaacggaaccgaaactatcaaacctccaatgaacggaaacccatacggacctaatccatctgacttcttgtccagagttaacaacttctcaatcatcgaaagtaccttgagagaaggagagcaattcgctaacgcctttttcgatactgagaagaaaattcagatcgcaaaggctcttgataacttcggagttgactacatcgaattgacttccccagttgcttcagaacaaagtagacaggactgtgaggctatctgcaaattgggtcttaagtgtaaaattttgactcatatcagatgccacatggatgacgctagagttgccgtcgaaactggtgttgatggagtcaacgttgtcattggtacatctcaatacttgagaaagtactctcatggtaaagatatgacctacatcatcgactctgctactgaggttattaattttgtcaagtccaaaggtatcgaagttagattttcttccgaggattctttcagatccgatcttgttgacttgctttctttgtacaaggctgttgataaaattggtgttaacagagtcggaatcgcagacaccgttggttgtgctactcctagacaagtctacgatcttatcagaacattgagaggagttgtctcctgtgatattgaatgccatttccacaacgacaccggtatggccattgcaaatgcttactgcgctttggaagccggagcaactcatatcgatacatcaatcttgggtatcggagagagaaatggtatcactccattgggagcattgcttgctagaatgtacgttacagatagagaatacatcacccacaagtacaaacttaaccaattgagagaacttgagaatttggttgctgacgccgttgaggtccagattccttttaacaattacatcactggaatgtgtgccttcacacataaggcaggtattcatgcaaaagctatccttgctaacccatcaacttacgaaattttgaagcctgaggatttcggaatgtcaagatatgttcatgtcggtagtagattgacaggatggaacgctattaagagtagagccgaacaattgaatcttcacttgacagacgcccaggcaaaagagttgaccgttagaattaagaaacttgccgatgtcagaactttggctatggatgacgttgacagagtcttgagagaataccacgcagacttgtccgacgcagatagaatcactaaggaagcctccgcctaattaattctgtctttgattttcttatgttattcaaaacatctgccccaaaatctaacgattatatatattcctacgtataactgtatagctaattattgatttatttgtacataaaaaccacataaatgtaaaagcaagaaaaaaaataactaaggagaaggatcaatatctcatttataatgctcgccaaagcagcgtacgtgaattttaatcaagacatcaacaaatcttgcaacttggttatatcgcttcttcacccactcacccgcttttctacattgttgaacacaaatatatacaggggtatgtctcaaggtcaagtgcagtttcaacagagactacctcaaggtacctcttcagaaatgcagaacttcactcttgatcagattttctccgaattaaaggtttaaacatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgttgag

SEQ ID NO: 19 - DNA, 2466 bp, Artificial Sequence
  Description of Artificial Sequence Synthetic C-term URA3 gene integration fragment
aattctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgcgtttggcgcgccatctaatagtttaatcacagcttatagtctactatagttttcttttttaaacattgttgtattttgtcccccccctctaattgatgatgattatcctataagaatccaataaaacgatggaaactaataccctctcctttgtcatgtggtctttagtatttcttgaacattggctctgatttctcgactttatagtcctattaaaatcgctgttagttctcgatcgttgtatctcgtttcttgtctctttggtggatgattttgcgtgcgaacatgtttttttccctttctctcaccatcatcgtgtagttcttgtcaccatcccccccaccccttccttctctcattgattctataagagcttatccacagaggtgcagtaacgaggtagtttaaccttcgagtggatcaaaatgtcacacaggcctgcggccgcatttggcaaggcgtatctatataggaggatcacaagaaaagagagttcaacttagggaaccaggcttgacaaaagataatattaaaaaaggaagaaaaagagagaaagaaagtaaagacaagaagaatcaactccaatttgaataaatgggcctgtaagaatcttcaatctcgatgacggggaaaacgtcctcttttatagaccaaccccttgtggggctgtcgggaaaagcgggtttcccgggaaactcatatctgattggggggttgaagctattccgcgttttggggggtgcggtttataagaagaatgagtaatgcaaacggcgtttataaagagaaagggtgtggcggtgttaagcggttgaggttttaaggtgtgggggacggggtgtgattttttcggcgttacgggcacggggtatgattctcttaatgtcatttccctttctttctacttctagggcgcgcagcgtgcgacaatggtgtttgttgtgcaaataggtggttgtgttgtcgttggagttctcatgaacatatcactctattagcaaatttaccctaagtatgtaaggtttagtgtgttgaggactaatacagacaatctttgtcttatatataaatggttctactcaattttataagttttttttttttttttttaatctctttatgaaaatcaagaatgacattaaaacaatcacaatattgtatgcaagaactttgcctctaaacctctttcaaagagatgcataattataaaacaaatctatttgcatattcgctttacacaactattcaagaatataattacctaaagagtccatcttgagttcaccaacaccactacttagagctcggtacccgc

SEQ ID NO: 20 - DNA, 3889 bp, Artificial Sequence
  Description of Artificial Sequence Synthetic GPD gene deletion fragment
ccttcatttacgaaataaagtgccgcggttacgcagcacacaccagcaatcacgtgcagtgtctttttcttttttttttcttttttttcctctttttcttttgttttgtttcgtttcttttccgccagttcccgttttccatttccggaacaacaatgggactccactgttttctttccccccttccgttttcggctcgcagtctgtacatgcacgtttatccgacacctgtcttgtttggcgcgtaattaatacagtttctccggagtccaggtctcggacgggtaatttacacgtcatcattcatttctgtgtcaagagaggtagcgcaaaaagtagaaatggtgaaccacgggaatgacttgctggaaatcgacgccagagtccatttgaaaacctacctctacaagagaggaaacacactacagggtgtccctggtccgtaaaatggcgtaatatgatgacttccctctatagacgttgtatttccagctccaacatggttaaactattgctatggtgatggtattacagatagtaaaagaaggaagggggggtggcaatctcaccctaacagttactaagaacgtctacttcatctactgtcaatatacattggccacatgccgagaaattacgtcgacgccaaagaagggctcagccgaaaaaagaaatggaaaacttggccgaaaagggaaacaaacaaaaaggtgatgtaaaattagcggaaaggggaattggcaaattgagggagaaaaaaaaaaggcagaaaaggaggcggaaagtcagtacgttttgaaggcgtcattggttttcccttttgcagagtgtttcatttcttttgtttcatgacgtagtggcgtttcttttcctgcactttagaaatctatcttttccttatcaagtaacaagcggttggcaaaggtgtatataaatcaaggaattcccactttgaaccctttgaattttgatatcgtttattttaaatttatttgcggccgcggatccctcgaggccttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgccccgggggatctggatccgcggccgcaataacctcagggagaactttggcattgtactctccattgacgagtccgccaacccattcttgttaaacctaaccttgcattatcacattccctttgaccccctttagctgcatttccacttgtctacattaagattcattacacattctttttcgtatttctcttacctccctcccccctccatggatcttatatataaatcttttctataacaataatatctactagagttaaacaacaattccacttggcatggctgtctcagcaaatctgcttctacctactgcacgggtttgcatgtcattgtttctagcagggaatcgtccatgtacgttgtcctccatgatggtcttcccgctgccactttctttagtatcttaaatagagcagatcttacgtccactgtgcatccgtgcaccccgaaaatcgtatggttttccttgccacctctcacaattttgaatatgctcaacgcgaaagagaggggaagaggaatcgcattcgtagagtggctacattcaaccctgacaaaggaactagcgtttgtgcaggagagagtggtttgcatagatttcctttcctttgcaagcatattatatagagtagccaatacagtaacagctacagcacaaaaaagagaacgagaacgagaacgagaacaagaacaagaactagcactactgtcactgccagcatcaacattactaccattattccaacatgtttgcaactagaaatataaccattggtgtcagaacactcagaccaaccagtttcttgaaaacaaggtcttttctgcaacagaggctacaatcaacgctaaagaagagctatgaaccaaccaaatccgagct

SEQ ID NO: 21 - DNA, 19 bp, Artificial Sequence
  Description of Artificial Sequence Synthetic primer
tggcccagggaatcattac

SEQ ID NO: 22 - DNA, 28 bp, Artificial Sequence
  Description of Artificial Sequence Synthetic primer
tcaccacctgtcagtgacgagccacttc

SEQ ID NO: 23 - DNA, 20 bp, Artificial Sequence
  Description of Artificial Sequence Synthetic primer
ggacccaatgcctcccaatc

SEQ ID NO: 24 - DNA, 21 bp, Artificial Sequence
  Description of Artificial Sequence Synthetic primer
cgtttctcccttccctgatag

SEQ ID NO: 25 - DNA, 3889 bp, Artificial Sequence
  Description of Artificial Sequence Synthetic GPD gene deletion fragment
ccttcatttacgaaataaagtgccgcggttacgcagcacacaccagcaatcacgtgcagtgtctttttcttttttttttcttttttttcctctttttcttttgttttgtttcgtttcttttccgccagttcccgttttccatttccggaacaacaatgggactccactgttttctttccccccttccgttttcggctcgcagtctgtacatgcacgtttatccgacacctgtcttgtttggcgcgtaattaatacagtttctccggagtccaggtctcggacgggtaatttacacgtcatcattcatttctgtgtcaagagaggtagcgcaaaaagtagaaatggtgaaccacgggaatgacttgctggaaatcgacgccagagtccatttgaaaacctacctctacaagagaggaaacacactacagggtgtccctggtccgtaaaatggcgtaatatgatgacttccctctatagacgttgtatttccagctccaacatggttaaactattgctatggtgatggtattacagatagtaaaagaaggaagggggggtggcaatctcaccctaacagttactaagaacgtctacttcatctactgtcaatatacattggccacatgccgagaaattacgtcgacgccaaagaagggctcagccgaaaaaagaaatggaaaacttggccgaaaagggaaacaaacaaaaaggtgatgtaaaattagcggaaaggggaattggcaaattgagggagaaaaaaaaaaggcagaaaaggaggcggaaagtcagtacgttttgaaggcgtcattggttttcccttttgcagagtgtttcatttcttttgtttcatgacgtagtggcgtttcttttcctgcactttagaaatctatcttttccttatcaagtaacaagcggttggcaaaggtgtatataaatcaaggaattcccactttgaaccctttgaattttgatatcgtttattttaaatttatttgcggccgcggatccagatcccccggggcgttgaagatctattctccagcaattaaatttgtgaagaataactggtatagagtacttcctttaaaaacatgtccgtgcaccaagaaaaaaaaaaagtttgaaaaattgtatgtcgacgaatttcagcattttcatttcaaggcgatattatgtttcactaaactcaggacaggaatatactaagaataactacaacatacacacaacataagccaagatggatcaacttaactaccaagaacaacaacaatttcaaaagatcgttgaacaaaagcaaatggctgatttcatgaggctatctgcagatacgcggaacaatcaatcgataatgatttgactgataaagaaaaccatacttttgtttatgtttattagttatcgctttgctacattaaaaattcacatactaaagcctttgttaaacaactttttctaaatcttaagattttactctatctagtttttttggttgtaggtgaacgtaaagtacctcatttatttttttttttttgcttgtgtaattcttttcatgcttatttaaactagtgtacatgtatcaaatctttgtgtaagaatcatttaaatctgtttaaataagcattccaaccagcttgttggtatcttttagcttgctctataggatctcttccttgaccgtacaaacctctaccaacaattatgatatccgttccagtctttacaacttcatcaacagttctatattgttgaccaagtgcatcacctttgtcatctaaaccaacccctggagtcataatgatccagtcaaaaccttcttctctaccgcccatatcgtgttgcgcaataaaaccaatgacaaactctttatcagatttagcaatttctactgttttttctgtatattcaccatatgctaaagaaccctttgatgataactcagcaagcattagcaaacctctaggttcactggttgtttcttgggctgcctccttcaagccagaaacaatacctgcacccgttacaccatgtgcattagtgatgtcagcccattcggcaatacggaagacaccagatttatattgatttttaacagtgttaccaatatcagcaaattttctatcttcaaaaatcataaaattatgtttcttggcaagctccttcaaaggcaacacagttccttcatacgtaaaatcagaaacaatatcgatgtgtgttttaactagacagatgtaaggaccaatagtgtccaaaatagagagaagcttttcagtttcagtaatatccaatgatgcacaaaggttagacttcttttcctccatgatggagaaaagtctcctagcaacaggggaagtgtgtgattctgatctttctttgtatgacgccatccttgacaaacaaactactttattaaagcgttgaagatctattctccagcaattaaatttgtgaagaataactggtatagagtacttcctttaaaaacatgtccgtgcaccaagaaaaaaaaaaagtttgaaaaattgtatgtcgacgaatttcagcattttcatttcaaggcgatattatgtttcactaaactcaggacaggaatatactaagaataactacaacatacacacaacataagccaagatggatcaacttaactaccaagaacaacaacaatttcaaaagatcgttgaacaaaagcaaatggctgatttcatgaggctatgaattcttttattataaaattatatattattcttaattacatatcacccttctatcagggaagggagaaacgaaaatagagagtgacctatccaagcttgggggtctaagttttaatggcccagggaatcattacttttttttctcaatccttgatggataaaagtattacatacgtacaggattgtgtattagtgtatttcgttatatgattaaacaaagtttatagattgtaaagtagacgtaaagtttagtaattcattttaatgttcattttacattcagatgttaattaaggcctcgagggatccgcggccgcaataacctcagggagaactttggcattgtactctccattgacgagtccgccaacccattcttgttaaacctaaccttgcattatcacattccctttgaccccctttagctgcatttccacttgtctacattaagattcattacacattctttttcgtatttctcttacctccctcccccctccatggatcttatatataaatcttttctataacaataatatctactagagttaaacaacaattccacttggcatggctgtctcagcaaatctgcttctacctactgcacgggtttgcatgtcattgtttctagcagggaatcgtccatgtacgttgtcctccatgatggtcttcccgctgccactttctttagtatcttaaatagagcagatcttacgtccactgtgcatccgtgcaccccgaaaatcgtatggttttccttgccacctctcacaattttgaatatgctcaacgcgaaagagaggggaagaggaatcgcattcgtagagtggctacattcaaccctgacaaaggaactagcgtttgtgcaggagagagtggtttgcatagatttcctttcctttgcaagcatattatatagagtagccaatacagtaacagctacagcacaaaaaagagaacgagaacgagaacgagaacaagaacaagaactagcactactgtcactgccagcatcaacattactaccattattccaacatgtttgcaactagaaatataaccattggtgtcagaacactcagaccaaccagtttcttgaaaacaaggtcttttctgcaacagaggctacaatcaacgctaaagaagagctatgaaccaaccaaatccgagct